(19) United States
(12) Patent Application Publication
(10) Pub. No.: US 2019/0365745 A1
(43) Pub. Date: Dec. 5, 2019

(54) USE OF CHRONIC TREATMENT WITH ATR INHIBITORS TO SENSITIZE CANCER CELLS TO PARP INHIBITORS

(71) Applicant: Cornell University, Ithaca, NY (US)

(72) Inventors: Marcus SMOLKA, Ithaca, NY (US); Dongsung KIM, Ithaca, NY (US)

(21) Appl. No.: 16/428,552

(22) Filed: May 31, 2019

Related U.S. Application Data
(60) Provisional application No. 62/679,340, filed on Jun. 1, 2018.

(51) Int. Cl. A61K 31/497; A61K 31/5377; A61K 31/4184; A61K 31/5025; A61K 31/451; A61K 31/55; A61K 31/502

(52) U.S. Cl. CPC A61K 31/497; A61K 31/5377; A61K 31/4184; A61K 31/502; A61K 31/451; A61K 31/55; A61K 31/5025

(57) ABSTRACT

The present application is directed to a method of sensitizing homologous recombination (HR)-proficient cancer cells to treatment with poly ADP ribose polymerase (PARP) inhibitors. This method involves providing HR-proficient cancer cells and treating the HR-proficient cancer cells with an Ataxia telangiectasia and Rad3-related (ATR) inhibitor under conditions effective to sensitize the HR-proficient cancer cells to treatment with PARP inhibitors, where the treating is carried out without administering PARP inhibitors. This technique can also be used to treat cancer in a subject. Additional methods of treating HR-proficient cancer cells and methods of treating a cancer patient are also disclosed.

This application claims the priority benefit of U.S. Provisional Patent Application Ser. No. 62/679,340, filed Jun. 1, 2018, which is hereby incorporated by reference in its entirety.

This invention was made with government support under R01GM097272 awarded by National Institute of Health. The government has certain rights in the invention.

### FIELD

The present application is directed to the use of chronic treatment with ATR inhibitors to sensitize cancer cells to PARP inhibitors.

### BACKGROUND

ATR (Ataxia telangiectasia and Rad3-related) is a member of the phosphatidylinositol-3-kinase-like kinase (PIKKs) family involved in genome maintenance. In response to DNA replication stress or DNA damage, ATR is activated and phosphorylates an extensive network of substrates, evoking a coordinated DNA damage response (Matsuoka et al., “ATM and ATR Substrate Analysis Reveals Extensive Protein Networks Responsive to DNA Damage,” Science 316:1160-1166 (2007); Smolka et al., “Proteome-wide Identification of in vivo Targets of DNA Damage Checkpoint Kinases,” Proc. Natl. Acad. Sci. USA 104:10364-10369 (2007); and Stokes et al., “Profiling of UV-Induced ATM/ATR Signaling Pathways,” Proc. Natl. Acad. Sci. USA 104:19855-19860 (2007)). While the related kinases ATM and DNA-PKcs are activated upon double strand breaks (DSBs), the ATR kinase specifically responds to exposure of single stranded DNA (ssDNA) resulting from a broad spectrum of DNA lesions (Zou et. al., “Sensing DNA Damage Through ATRIP Recognition of RPA-ssDNA Complexes,” Science 300:1542-1548 (2003)). Upon replication stress or detection of replication-associated lesions, ATR is recruited to RPA-coated ssDNA and becomes activated through the action of the ATR activators TOPBP1 and ETAA1 (Bass et al., “ETAA1 Acts at Stalled Replication Forks to Maintain Genome Integrity,” Nat. Cell Biol. 18:1185-1195 (2016); Haahr et al., P., “Activation of the ATR Kinase by the RPA-Binding Protein ETAA1,” Nat. Cell Biol. 18:1196-1207 (2016); Kumagai et al. “TopBP1 Activates the ATR-ATRIP Complex,” Cell 124:943-955 (2006); Lee et al., “RPA-Binding Protein ETAA1 Is an ATR Activator Involved in DNA Replication Stress Response,” Curr. Biol. 26:3257-3268 (2016); Mordes et al., “Dpb11 Activates the Mec1-Ddc2 Complex,” Proc. Natl. Acad. Sci. USA 105:18730-18734 (2008); and Lin et al., “The Rad4(TopBP1) ATR-Activation Domain Functions in G1/S Phase in a Chromatin-Dependent Manner,” PLoS Genet. 8:e1002801 (2012)). In response to replication stress, ATR has been shown to mediate a global cellular response that promotes cell cycle arrest, inhibition of late origin firing, stabilization of replication forks, transcriptional regulation and DNA repair (Nam et al., “ATR Signalling: More than Meeting at the Fork,” Biochem. J. 436:527-536 (2011) and Zeman et al., “Causes and Consequences of Replication Stress,” Nat. Cell Biol. 16:2-9 (2014)). ATR kinase exerts its function in genome maintenance by targeting and phosphorylating the key effector kinase CHK1, which mediates cell cycle arrest through the phosphorylation and degradation of the CDC25 phosphatase (Xiao et al., “Chk1 mediates S and G(2) arrests through Cdc25A degradation in response to DNA-damaging agents,” J. Biol. Chem. 278:21767-21773 (2003); Jin et al., “SCF beta-TRCP Links Chk1 Signaling to Degradation of the Cdc25A Protein Pphosphatase,” Gene Dev. 17:3062-3074 (2003); and Sorensen et al., “Chk1 Regulates the S Phase Checkpoint by Coupling the Physiological Turnover and Ionizing Radiation-Induced Accelerated Proteolysis of Cdc25A,” Cancer Cell 3:247-258 (2003)). In addition, ATR-CHK1 signaling plays a prominent role in controlling E2F-dependent transcription (Bertoli et al., “Control of Cell Cycle Transcription During G1 and S Phases,” Nat. Rev. Mol. Cell Biol. 14:518-528 (2013); Buisson et al., “Distinct but Concerted Roles of ATR, DNA-PK, and Chk1 in Countering Replication Stress during S Phase,” Mol. Cell 59:1011-1024 (2015); and Bertoli et al., “Sustained E2F-Dependent Transcription Is a Key Mechanism to Prevent Replication-Stress-Induced DNA Damage. Cell Rep. 15:1412-1422 (2016)), which includes a large set of genes with important roles in DNA replication, DNA repair and cell cycle control (Bracken et al., “E2F Target Genes: Unraveling the Biology,” Trends Biochem. Sci. 29: 409-417 (2004)). Mechanistically, CHK1 has been shown to phosphorylate and inhibit the E2F6 repressor (Bertoli et al., “Chk1 Inhibits E2F6 Repressor Function in Response to Replication Stress to Maintain Cell-Cycle Transcription,” Curr. Biol. 23:1629-1637 (2013)). Additional mechanisms may also couple ATR and CHK1 to the control of E2F-dependent transcription (Bertoli et al., “Control of Cell Cycle Transcription During G1 and S Phases,” Nat. Rev. Mol. Cell Biol. 14:518-528 (2013) and Lin et al., “Selective Induction of E2F1 in Response to DNA Damage, Mediated by ATM-Dependent Phosphorylation,” Genes Dev. 15:1833-1844 (2001)).

ATR also plays crucial roles in the control of DNA repair. It has been shown that ATR signaling regulates the repair of DNA interstrand cross-links and nucleotide excision repair by directly phosphorylating Fanconi Anemia (FA) or Xeroderma Pigmentosum (XP) proteins (Collis et al., “FANCM and FAAP24 Function in ATR-Mediated Checkpoint Signaling Independently of the Fanconi Anemia Core Complex,” Mol. Cell 32:313-324 (2008); Collins et al., “ATR-Dependent Phosphorylation of FANCA on Serine 1449 after DNA Damage is Important for FA Pathway Function,” Blood 113:2181-2190 (2009); and Wang et al., “The Fanconi Anemia Pathway and ICL Repair: Implications for Cancer Therapy.” Crit. Rev. Biochem. Mol. Biol. 45:424-439 (2010)). In addition, the roles for ATR in homologous recombination (HR)-mediated repair have recently been proposed as a crucial pathway to repair DSBs (Liu et al., “TOPBP1(Dpb11) Plays a Conserved Role in Homologous Recombination DNA Repair Through the Coordinated Recruitment of 53BP1(Rad9),” J. Cell Biol. 216:623-639 (2017); Buisson et al., “Coupling of Homologous Recombination and the Checkpoint by ATR,” Mol. Cell 65:336-346 (2017); and Wang et al., “ATR Affecting Cell Radiosensitivity is Dependent on Homologous Recombination Repair but Independent of Nonhomologous End Joining,” Cancer Res. 64:7139-7143 (2004)). Of note, HR-mediated repair occurs preferably during S/G2 phase of the cell cycle since sister chromatids can be used as a template for error-free DNA repair (Aylon et al., “The CDK Regulates Repair of Double-Strand Breaks by Homologous Recombination During the Cell Cycle,” EMBO J. 23:4868-4875 (2004); Ira et al., “DNA End Resection, Homologous Recombination and DNA Damage Checkpoint Activation Require CDK1,” Nature 431:1011-1017 (2004); and Branzei et al., “Regulation of DNA Repair Throughout the Cell Cycle,” Nat. Rev. Mol. Cell Biol. 9:297-308 (2008)). As an alternative to HR, cells may repair DSBs using non-homologous end joining (NHEJ), which is relatively less favored in S/G2 as compared to in the G1 phase of the cell cycle (Branzei et al., “Regulation of DNA Repair Throughout the Cell Cycle,” Nat. Rev. Mol. Cell Biol. 9:297-308 (2008) and Heidenreich et al., “Non-Homologous End Joining as an Important Mutagenic Process in Cell Cycle-Arrested Cells,” EMBO J. 22:2274-2283 (2003)). Since the improper use of NHEJ in S phase leads to chromosomal aberrations and decreased survival (Bunting et al., “53BP1 Inhibits Homologous Recombination in Brca1-Deficient Cells by Blocking Resection of DNA Breaks,” Cell 141:243-254 (2010) and Escribano-Diaz et. al., “A Cell Cycle-Dependent Regulatory Circuit Composed of 53BP1-RIF1 and BRCA1-CtIP Controls DNA Repair Pathway Choice,” Mol. Cell 49:872-883 (2013)), balanced engagement of HR and NHEJ repair pathways is essential for maintaining genomic integrity. Recently, ATR was shown to promote HR by phosphorylating PALB2 and enhancing its localization to DNA lesions via an interaction with BRCA1 (Buisson et al., “Coupling of Homologous Recombination and the Checkpoint by ATR,” Mol. Cell 65:336-346 (2017)). Additionally, it has been proposed that ATR mediates BRCA1 phosphorylation and its interaction with TOPBP1 to promote HR by stabilizing BRCA1 at lesions during S-phase (Liu et al., “TOPBP1(Dpb11) Plays a Conserved Role in Homologous Recombination DNA Repair Through the Coordinated Recruitment of 53BP1(Rad9),” J. Cell Biol. 216:623-639 (2017)). Therefore, ATR seems to play a key role in promoting HR-mediated repair and suppressing improper NHEJ during replication stress.

The physiological importance of ATR is highlighted by the fact that mice lacking functional ATR are embryonic lethal (Brown et al., “ATR Disruption Leads to Chromosomal Fragmentation and Early Embryonic Lethality,” Genes Dev. 14:397-402 (2000) and Liu et al., “Chk1 is an Essential Kinase that is Regulated by Atr and Required for the G(2)/M DNA Damage Checkpoint,” Genes Dev. 14:1448-1459 (2000)). Also, homozygous mutations in human ATR that cause defective mRNA splicing and severely reduced ATR expression are associated with Seckel syndrome, a genetic disorder characterized by growth defect (dwarfism), microcephaly and mental retardation (O'Driscoll et al., “A Splicing Mutation Affecting Expression of Ataxia-Telangiectasia and Rad3-Related Protein (ATR) Results in Seckel Syndrome,” Nat. Genet. 33:497-501 (2003)). Notably, Seckel syndrome cells show high genomic instability and increased micronuclei formation (Casper et al., “Chromosomal Instability at Common Fragile Sites in Seckel Syndrome,” Am. J. Hum. Genet. 75:654-660 (2004) and Alderton et al., “Seckel Syndrome Exhibits Cellular Features Demonstrating Defects in the ATR-Signalling Pathway,” Hum. Mol. Genet. 13:3127-3138 (2004)), supporting the role of ATR in genome integrity.

In the context of cancer, ATR is believed to be crucial for the ability of many cancer types to withstand the increased levels of replication stress generated by oncogene-induced de-regulation of DNA replication (Bertoli et al., “Sustained E2F-Dependent Transcription Is a Key Mechanism to Prevent Replication-Stress-Induced DNA Damage,” Cell Rep. 15:1412-1422 (2016); Karnitz et al., “Molecular Pathways: Targeting ATR in Cancer Therapy,” Clin. Cancer Res. 21:4780-4785 (2015); Herlihy et al., “The Role of the Transcriptional Response to DNA Replication Stress,” Genes (Basel) 8 (2017); Hills et al., “DNA Replication and Oncogene-Induced Replicative Stress,” Current Biology 24:R435-R444; and Liu et al., “The ATR-Mediated S Phase Checkpoint Prevents Rereplication in Mammalian Cells when Licensing Control is Disrupted,” J. Cell Biol. 179:643-657 (2007)). While the inhibition of ATR activity leads to moderate cytotoxicity in normal cells due to increased fork stalling and collapse, this cytotoxicity is further exacerbated in cancer cells with higher replication stress, providing rationale for using ATR inhibitors (ATRi) in cancer treatment (Gilad et al., “Combining ATR Suppression with Oncogenic Ras Synergistically Increases Genomic Instability, Causing Synthetic Lethality or Tumorigenesis in a Dosage-Dependent Manner,” Cancer Res. 70:9693-9702 (2010) and Syljuasen et al., “Targeting Lung Cancer Through Inhibition of Checkpoint Kinases,” Frontiers in Genetics 6 (2015)). Cancer cells frequently bear mutations in components of DNA damage response pathways, leading to increased dependency on ATR signaling (Weber et al., “ATM and ATR as Therapeutic Targets in Cancer,” Pharmacol. Therapeut. 149:124-138 (2015)). Consistent with this notion, it has been shown that inhibition of ATR kinase activity is synthetic lethal in tumor cells that have mutations in ATM, p53, ERCC1 and XRCC1 (Mohni et al, “A Synthetic Lethal Screen Identifies DNA Repair Pathways that Sensitize Cancer Cells to Combined ATR Inhibition and Cisplatin Treatments,” PLoS One 10:e0125482 (2015); Pires et al., “Targeting Radiation-Resistant Hypoxic Tumour Cells Through ATR Inhibition,” Brit. J. Cancer 107:291-299 (2015); Reaper et al., Selective Killing of ATM- or p53-Deficient Cancer Cells Through Inhibition of ATR,” Nat. Chem. Biol. 7:428-430 (2011); Josse et al., “ATR Inhibitors VE-821 and VX-970 Sensitize Cancer Cells to Topoisomerase i Inhibitors by Disabling DNA Replication Initiation and Fork Elongation Responses,” Cancer Res. 74:6968-6979 (2014); Huntoon et al., “ATR Inhibition Broadly Sensitizes Ovarian Cancer Cells to Chemotherapy Independent of BRCA Status,” Cancer Res. 73:3683-3691 (2013); Mohni et al., “ATR Pathway Inhibition Is Synthetically Lethal in Cancer Cells with ERCC1 Deficiency,” Cancer Res. 74:2835-2845 (2014); and Sultana et al., “Ataxia Telangiectasia Mutated and Rad3 Related (ATR) Protein Kinase Inhibition is Synthetically Lethal in XRCC1 Deficient Ovarian Cancer Cells,” PLoS ONE 8:e57098 (2013)). Therefore, specific inhibition of ATR signaling is expected to selectively kill cancer cells with genetic defects in DNA damage response pathways and/or elevated oncogene-induced replication stress. Accordingly, in the last eight years, highly selective and potent ATR inhibitors have been developed and are currently under phase I/II clinical trials in cancer treatment (Karnitz et al., “Molecular Pathways: Targeting ATR in Cancer Therapy,” Clin. Cancer Res. 21:4780-4785 (2015) and Weber et al., “ATM and ATR as Therapeutic Targets in Cancer,” Pharmacol. Therapeut. 149: 124-138 (2015)).

Despite extensive work establishing ATR inhibitors as potential anti-cancer agents, it is not yet well understood precisely how ATR inhibition impacts DNA repair in the context of normal and cancer cells.

The present application is directed to overcoming these and other deficiencies in the art.

### SUMMARY

One aspect of the present application relates to a method of sensitizing homologous recombination (HR)-proficient cancer cells to treatment with poly ADP ribose polymerase (PARP) inhibitors. This method involves providing HR-proficient cancer cells and treating the HR-proficient cancer cells with an Ataxia telangiectasia and Rad3-related (ATR) inhibitor under conditions effective to sensitize the HR-proficient cancer cells to treatment with PARP inhibitors, where the treating is carried out without administering PARP inhibitors.

Another aspect of the present application relates to a method of treating cancer in a subject. This method involves selecting a subject with a cancer mediated by homologous recombinant (HR)-proficient cells and treating the selected subject with an Ataxia telangiectasia and Rad3-related (ATR) inhibitor under conditions effective to sensitize the HR-proficient cancer cells to treatment with PARP inhibitors, where the treating is carried out without administering PARP inhibitors.

A further aspect of the present application relates to a method of treating HR-proficient cancer cells. This method involves providing HR-proficient cancer cells; treating the HR-proficient cancer cells with an ATR inhibitor, where the treating is carried out without administering PARP inhibitors; and administering to the treated HR-proficient cancer cells an ATR inhibitor and a PARP inhibitor.

Yet another aspect of the present application relates to a method of treating a cancer patient. This method involves selecting a subject with a cancer mediated by HR-proficient cells; treating the selected subject with an ATR inhibitor, where the treating is carried out without administering PARP inhibitors; and administering to the selected subject an ATR inhibitor and a PARP inhibitor.

As described herein, ATR has been shown to control HR-mediated repair, however, the effects of short-term treatment with ATR inhibitors on HR are rather modest. The present disclosure demonstrate that chronic ATR inhibition severely impairs the ability of cancer cells to utilize HR-mediated repair, leading to an effect that is significantly stronger when compared to acute ATR inhibition. Proteomic analysis reveals that chronic, but not acute, ATR inhibition depletes the abundance of key components of the HR machinery, including TOPBP1, BRCA1, and RAD51. The results presented herein demonstrate that ATR-mediated control of HR factor abundance involves transcription and post-translational control mechanisms. Interestingly, cancer cells seem to exhibit a stronger dependency on ATR signaling for maintaining the levels of HR factors, providing rationale for the use of ATR inhibitors in combination with drugs known to preferentially target HR-deficient cells for anti-cancer therapy.

### BRIEF DESCRIPTION OF THE DRAWINGS

FIGS. 1A-1D demonstrate that chronic inhibition of ATR impairs HR-mediated repair in U2OS cells. FIG. 1A is a bar graph showing homologous recombination (HR) efficiency measured in U2OS cells using the DR-GFP reporter system. Relative fold change of GFP positive cells in ATR inhibitor (ATRi) or ATM inhibitor (ATMi) treated cells over DMSO. (Error bars=SEM, N≥5, ****P-value<0.0001; one sample-t-test was used for the statistical analysis). FIG. 1B is a bar graph showing non-homologous end joining (NHEJ) efficiency measured in U2OS cells using the EJ5-GFP reporter system. Relative fold change of GFP positive cells in ATRi or ATMi treated cells over DMSO. (Error bars=SEM, N=5, one sample-t-test was used for the statistical analysis). FIGS. 1C-1D show representative FACS data (FIG. 1C) and quantitation (FIG. 1D) of G1, S and G2/M population for cell cycle analysis of ATRi treated cells (Error bars=SD, N=3).

FIGS. 2A-2B show the results of ATR inhibition in U2OS cells. FIG. 2A are histograms showing representative flow-cytometric DR-GFP analysis relative to FIG. 1A. U2OS cells were treated with DMSO (8 days) or ATRi (2 days). Cells transfected with DR-GFP+pCAGGS, DR-GFP+ISceI and mCherry are shown. FIG. 2B are histograms showing representative flow-cytometric DR-GFP analysis relative to FIG. 1A. U2OS cells were treated with DMSO (8 days) or ATRi (8 days) or ATMi (8 days). Cells transfected with DR-GFP+pCAGGS, DR-GFP+ISceI and mCherry are shown.

FIG. 3 is a Western Blot (“WB”) of U2OS cells treated with ATRi for 30 minutes prior to a 3-hour HU treatment. WB analysis was performed against phospho-CHK1(S345), CHK1 and Actin.

FIG. 4 are histograms showing representative flow-cytometric EJ5-GFP analysis relative to FIG. 1B. U2OS cells were treated with DMSO (8 days) or ATRi (8 days). Cells transfected with EJ5-GFP+pCAGGS, EJ5-GFP+ISceI and mCherry are shown.

FIGS. 5A-F demonstrate that chronic inhibition of ATR depletes the abundance of key HR factors. FIG. 5A is a schematic diagram of the SILAC/MS experiment. FIG. 5B is a quadrant diagram of the SILAC/MS experiment. Each dot represents a different identified protein. Proteins with abundance reduced two-fold or more after ATRi treatment are shown in the upper-left corner (white quadrant). FIG. 5C is a WB analysis of U2OS cells after 8 days of chronic treatment with 5 μM of ATRi or ATMi. FIG. 5D is a time course WB analysis of U2OS cells during chronic treatment with 5 μM ATRi. FIG. 5E is a WB analysis of U2OS cells at distinct cell cycle stages. Cells were treated with 1 mM hydroxyurea (HU) or 50 ng/ml nocodazole (Noc) for 24 hours. HU treated cells were released for 2.5 hours in HU-free media. FIG. 5F is a WB analysis of U2OS cells untreated or treated for 8 days with 5 μM ATRi.

FIG. 6 is a table showing GO-Analysis of FIG. 5B. The top 22 categories significantly enriched in the group of proteins with reduced abundance upon chronic ATRi treatment are shown. Of note, the category of “cell cycle” seems artificially over-represented since many bona-fide repair and replication proteins are also considered as part of the “cell cycle” category.

FIG. 7 shows cell cycle analysis of cells from FIG. 5E.

FIGS. 8A-8D show that fibroblasts derived from Seckel syndrome patient have reduced abundance and impaired localization of HR factors. FIGS. 8A-8B show WB (FIG. 8A) and FACS (FIG. 8B) analysis of untreated Seckel syndrome and healthy control (non-isogenic) cells. FIG. 8C shows immunofluorescence analysis of Seckel syndrome and healthy control cells treated with 10 Gy γ-irradiation and fixed after 8 hours of recovery. Staining was performed with RAD51 or BRCA1 antibody. (Scale bar=10 μm). FIG. 8D is a bar graph showing the quantitation of immunofluorescence results. Error bars=SEM, N≥3, **P-value<0.01; two-tailed Student's t-test was used for the statistical analysis.

FIGS. 9A-9J show that ATR controls HR factor abundance via CHK1-mediated transcription. FIG. 9A shows WB analysis of U2OS cells after chronic treatment with CHK1 inhibitor (0.2 μM for 5 days). FIG. 9B is a bar graph showing the analysis of HR-mediated repair using the DR-GFP system. Relative fold change of GFP positive U2OS cells treated with CHK1i compared to DMSO treatment control. (Error bars=SEM, N=4, ****P-value<0.0001; one sample-t-test was used for the statistical analysis). FIGS. 9C-9D show a histogram showing representative FACS data (FIG. 9C) and a bar graph showing quantitation of G1, S and G2/M population for cell cycle analysis of CHK1i treated cells (FIG. 9D) (Error bars=SD, N=3). FIG. 9E are bar graphs showing real time PCR analysis of total RNA extracted from U2OS cells treated with DMSO, ATRi (5 μM for 8 days) or CHK1i (0.2 μM for 5 days). Values were normalized to GAPDH mRNA levels. (Error bars=SD, N=3). FIG. 9F is a schematic diagram showing a proposed model of how E2F transcription is regulated through ATR-CHK1 signaling. FIG. 9G shows WB analysis of 293-T-REX-E2F6 cells after 8 days of treatment with DMSO or doxycycline. FIG. 9H is a bar graph showing DR-GFP analysis of 293-T-REX-E2F6 cells after 8 days of treatment with DMSO or doxycycline. (Error bars=SEM, N=4). FIG. 9I is a histogram showing cell cycle analysis of 293-T-REX-E2F6 cells after 8 days of treatment with DMSO or doxycycline. FIG. 9J is a schematic diagram showing a model depicting multiple roles of ATR in the control of HR capacity.

FIG. 10 is a dot plot showing representative flow-cytometric DR-GFP analysis relative to FIG. 9B. U2OS cells were treated with DMSO or CHK1i for 5 days. Cells transfected with DR-GFP+pCAGGS, DR-GFP+ISceI and mCherry are shown.

FIG. 11 shows the evaluation of U2OS cells treated with cycloheximide. The left panel shows WB analysis of U2OS cells treated with cycloheximide (CHX, 50 μg/ml) with or without 5 μM of ATRi. Fresh media containing CHX or CHX/ATRi was added every 6 hours. The right panel is a bar graph generated by measuring the intensity of the BRCA1 band from three independent experiments. Two-tailed Student's t-test was used for the statistics (Error bars=SD, N=3, ****P-value<0.0001).

FIGS. 12A-12F show the correlation between HR factor abundance and HR-mediated repair. FIG. 12A shows WB analysis of HR factor abundance in a panel of cell lines. FIG. 12B is a bar graph showing the analysis of HR-mediated repair using the DR-GFP system. Relative fold change of GFP positive population from different cell lines (Error bars=SEM, N≥3). FIG. 12C shows graphs showing R2 correlation between protein abundance and DR-GFP level. The abundance of each protein was measured by densitometry and normalized to corresponding abundance of actin. FIG. 12D is a diagram of the experimental setup and rationale for using LT transformation to induce increased replication stress, HR factor abundance and HR capacity. FIG. 12E shows WB analyses of hTERT RPE-1 and LT transformed hTERT RPE-1 cells. FIG. 12F is a bar graph showing an analysis of HR-mediated repair using the DR-GFP system. Relative fold change of GFP positive hTERT RPE-1 cells over LT transformed hTERT RPE-1 cells (Error bars=SEM, N≥3, ****P-value<0.0001; one sample-t-test was used for the statistical analysis).

FIG. 13 shows cell cycle analysis of hTERT RPE-1 and hTERT RPE-1/LT. (Error bars=SD, N=3). The left panel shows histograms of hTERT RPE-1 and hTERT/LT RPE-1. The right panel shows a bar graph of the percentage of cells in G1, G, and G2/M.

FIGS. 14A-14E show that cancer cell lines display high dependency on ATR signaling for sustaining the abundance of HR factors and HR-mediated repair. FIGS. 14A-14B show WB analysis of hTERT RPE-1 (FIG. 14A) or HCT116 (colon cancer)(FIG. 14B) cells after 8 day chronic treatment with 5 μM ATRi or ATMi. FIG. 14C shows WB analysis of indicated cancer cell lines upon 8 day chronic treatment with 5 μM ATRi. FIGS. 14D-14E are bar graphs showing the analysis of HR-mediated repair using the DR-GFP system. Relative fold change of GFP positive hTERT RPE-1 (FIG. 14D) or HCT116 (FIG. 14E) cells over DMSO after ATRi treatment. (Error bars=SEM, N≥3, ****P-value<0.0001; one sample-t-test was used for the statistical analysis comparing 2 days versus 8 days of treatment).

FIGS. 15A-15B show the results of ATR inhibition in hTERT RPE-1 cells. FIG. 15A shows dot plots of a representative flow-cytometric DR-GFP analysis of FIG. 14D. RPE-1 cells were treated with DMSO (8 days) or ATRi (2 or 8 days). Cells transfected with DR-GFP+pCAGGS, DR-GFP+ISceI and mCherry are shown. FIG. 15B shows cell cycle analysis of RPE-1 cells treated with ATRi for 8 days. (Error bars=SD, N=3)

FIGS. 16A-16B show that chronic treatment with ATR inhibitor hypersensitizes several HR-proficient cancer cells, but not hTERT RPE-1 cells, to PARP inhibition. FIG. 16A shows cell viability analysis for testing the synergistic effect of ATRi and PARPi following chronic treatment with ATRi. First, cells were treated for 5 days with either DMSO or 5 μM ATR inhibitor. Next, 1×10⁵ cells of each cell type were plated onto a 10 cm dish and further treated with DMSO, ATR inhibitor and/or PARP inhibitor for additional 3 days. Cells were either fixed or allowed to recover after additional 2 or 8 days in drug-free media. Crystal violet staining of representative results are shown at the top and quantitation of viable cells from multiple experiments is shown at the bottom. (Error bars=SD, N=3). FIG. 16B shows bar graphs of the indicated cancer cell lines subjected to the same experimental protocol described in FIG. 16A. These cells did not display noticeable sensitivity to treatment with PARP inhibitor only (FIG. 18A).

FIGS. 17A-17B show the results of ATRi inhibition in HCT116 cells. FIG. 17A show dot plots of representative flow-cytometric DR-GFP analysis of FIG. 14E. HCT116 cells were treated with DMSO (8 days) or ATRi (2 or 8 days). Cells transfected with DR-GFP+pCAGGS, DR-GFP+ISceI and mCherry are shown. FIG. 17B shows cell cycle analysis of HCT116 cells treated with ATRi for 8 days. (Error bars=SD, N=3).

FIGS. 18A-18B show the results of cells treated with PARPi and/or ATRi. FIG. 18A shows representative crystal violet images of HCT116, HeLa, A2780 and U87 cancer cells treated with DMSO or PARPi for 3 days. Cells were treated with DMSO for 5 days prior to any treatment. FIG. 18B shows representative crystal violet images of HCT116, HeLa, A2780 and U87 cancer cells recovered for 2 days after 3 days of ATRi or ATRi+PARPi treatment. Cells were treated with ATRi for 5 days prior to any treatment to allow reduction of HR factors. Please see FIG. 7B for more information.

FIGS. 19A-19C show a model depicting how modulation of ATR signaling alters HR capacity in cancer cell growth and ATRi-mediated cancer therapy. FIG. 19A is a model of steady state HR capacity. FIG. 19B is a model of ATR inhibitor-induced decrease in HR capacity. FIG. 19C is a model of replication stress-induced increase in HR capacity.

FIG. 20 shows WB analysis of HCT116 cells after chronic CHK1i.

### DETAILED DESCRIPTION

In this specification and the appended claims, the singular forms “a”, “an”, and “the” include plural references unless the context clearly dictates otherwise.

The terms “comprising”, “comprises”, and “comprised of”, as used herein, are synonymous with “including”, “includes” or “containing”, “contains”, and are inclusive or open-ended and do not exclude additional, non-recited members, elements, or method steps.

The recitation of numerical ranges by endpoints includes all numbers and fractions subsumed within the respective ranges, as well as the recited endpoints.

One aspect of the present application described herein relates to a method of sensitizing homologous recombination (HR)-proficient cancer cells to treatment with poly ADP ribose polymerase (PARP) inhibitors. This method involves providing HR-proficient cancer cells and treating the HR-proficient cancer cells with an Ataxia telangiectasia and Rad3-related (ATR) inhibitor under conditions effective to sensitize the HR-proficient cancer cells to treatment with PARP inhibitors, where the treating is carried out without administering PARP inhibitors.

Another aspect of the present application relates to a method of treating cancer in a subject. This method involves selecting a subject with a cancer mediated by homologous recombinant (HR)-proficient cancer cells and treating the selected subject with an Ataxia telangiectasia and Rad3-related (ATR) inhibitor under conditions effective to sensitize the HR-proficient cancer cells to treatment with PARP inhibitors, where the treating is carried out without administering PARP inhibitors.

As used herein, the terms “cancer” and “cancerous” refer to or describe the physiological condition in which a population of cells are characterized unregulated cell growth. Examples of cancer include, but are not limited to, carcinoma, sarcoma, melanoma, leukemia, lymphoma, and combinations thereof (mixed-type cancer). A “carcinoma” is a cancer originating from epithelial cells of the skin or the lining of the internal organs. A “sarcoma” is a tumor derived from mesenchymal cells, usually those constituting various connective tissue cell types, including fibroblasts, osteoblasts, endothelial cell precursors, and chondrocytes. A “melanoma” is a tumor arising from melanocytes, the pigmented cells of the skin and iris. A “leukemia” is a malignancy of any of a variety of hematopoietic stem cell types, including the lineages leading to lymphocytes and granulocytes, in which the tumor cells are nonpigmented and dispersed throughout the circulation. A “lymphoma” is a solid tumor of the lymphoid cells. More particular examples of such cancers include, e.g., acinar cell carcinoma, adenocarcinoma (ductal adenocarcinoma), adenosquamous carcinoma, anaplastic carcinoma, cystadenocarcinoma, duct-cell carcinoma (ductal adrenocarcinoma), giant-cell carcinoma (osteoclastoid type), mixed-cell carcinoma, mucinous (colloid) carcinoma, mucinous cystadenocarcinoma, papillary adenocarcinoma, pleomorphic giant-cell carcinoma, serous cystadenocarcinoma, and small-cell (oat-cell) carcinoma. As used herein, cancers are named according to the organ in which they originate.

The terms “cancer cell” and “tumor cell” refer to one or more cells derived from a tumor or cancerous lesion.

Malignant tumors are distinguished from benign growths or tumors in that, in addition to uncontrolled cellular proliferation, they can invade surrounding tissues and can metastasize. The term “metastasis” or “metastasize” as used herein refers to a process in which cancer cells travel from one organ or tissue to another non-adjacent organ or tissue. In some embodiments, the cancer cells are metastatic cancer cells or metastatic tumor cells.

In the context of the methods described herein, the cancer cells may be selected from the group consisting of osteosarcoma cells, colon cancer cells, ovarian cancer cells, cervical cancer cells, glioblastoma cells, neuroblastoma cells, lung cancer cells, and pancreatic cancer cells.

Osteosarcoma, also known as osteogenic sarcoma, is the most common type of bone cancer and typically starts in bone cells in the arms, legs, or pelvis. Osteosarcomas may be classified as high-grade, intermediate-grade, and low-grade osteosarcomas. Exemplary high-grade osteosarcomas include, but are not limited to, osteoblastic, chondroblastic, fibroblastic, small cell, telangiectatic, high-grade surface (juxtacortical high grade), pagetoid, extraskeletal, and post-radiation osteosarcomas. Thus, in some embodiments, the osteosarcoma cells selected from the group consisting of osteoblastic osteosarcoma cells, chondroblastic osteosarcoma cells, fibroblastic osteosarcoma cells, small cell osteosarcoma cells, telangiectatic osteosarcoma cells, high-grade surface (juxtacortical high grade) osteosarcoma cells, pagetoid osteosarcoma cells, extraskeletal osteosarcoma cells, and post-radiation osteosarcoma cells. Exemplary intermediate-grade osteosarcomas include, e.g., periosteal (juxtacortical intermediate grade) osteosarcomas. Thus, in some embodiments, the osteosarcoma cells are periosteal osteosarcoma cells. Exemplary low-grade osteosarcomas include, e.g., parosteal (juxtacortical low grade) and intramedullary or intraosseous well differentiated (low-grade central). Thus, in some embodiments, the osteosarcoma cells are parosteal osteosarcoma cells, intramedullary osteosarcoma cells, or intraosseous well differentiated osteosarcoma cells.

The large intestine comprises the cecum, colon, rectum, and anal canal. The colon receives almost completely digested food from the cecum, absorbs water and nutrients, and passes waste to the rectum. Exemplary colon cancers include, but are not limited to, adenocarcinoma, carcinoid tumors, gastrointestinal stromal tumors, lymphomas, and sarcomas. The rectum receives waste from the colon and stores it until it passes out of the body through the anus. Exemplary rectal cancers include, but are not limited to, adenocarcinoma, carcinoid tumors, gastrointestinal stromal tumors, lymphomas, and sarcomas. The cancer cells may be rectal cancer cells.

The ovaries comprise epithelial cells, germ cells, and stromal cells. Exemplary ovarian cancers include, e.g., epithelial tumors (carcinomas), germ cell tumors, and stromal tumors. Exemplary ovarian carcinomas include, but are not limited to, serous carcinoma, clear cell carcinoma, mucinous carcinoma, and endometrioid carcinoma. Thus, in some embodiments, the ovarian cancer cells are selected from the group consisting of serous carcinoma cells, clear cell carcinoma cells, mucinous carcinoma cells, and endometrioid carcinoma cells. Exemplary germ cell tumors include, but are not limited to teratomas, dysgerminomas, endodermal sinus tumors, and choriocarcinomas. Thus, in some embodiments, the ovarian cancer cells are selected from the group consisting of teratoma cells, dysgerminoma cells, endodermal sinus tumor cells, and choriocarcinoma cells. Exemplary ovarian stromal tumors include, but are not limited to, granulosa cell tumors, granulosa-theca tumors, and Sertoli-Leydig cell tumors. Thus, in some embodiments, the ovarian cancer cells are selected from the group consisting of granulosa cell tumor cells, granulosa-theca tumor cells, and Sertoli-Leydig cell tumor cells.

The cervix connects the body of the uterus to the vagina. Exemplary cervical cancers include, but are not limited to squamous cell carcinomas, adenocarcinomas, adenosquamous carcinomas (mixed carcinomas), melanomas, sarcomas, and lymphomas. Thus, in some embodiments, the cervical cancer cells are selected from the group consisting of squamous cell carcinoma cells, adenocarcinoma cells, adenosquamous carcinoma (mixed carcinomas) cells, melanoma cells, sarcoma cells, and lymphoma cells.

Glioblastomas are the most common malignant brain tumor in adults. Glialblastomas originate in glial cells called astrocytes (astrocytomas). Additional exemplary astrocytomas include, e.g., non-infiltrating astroctyomas, low-grade astrocytomas, and anaplastic astroctyomas. In some embodiments, the cancer cells are glioblastoma cells.

Neuroblastomas occur most often in infants and young children and originate in neuroblast cells. In some embodiments, the cancer cells are neuroblastoma cells.

Exemplary lung cancers include, but are not limited to, non-small cell lung cancer, small cell lung cancer, and lung carcinoid tumors. Non-small cell lung cancer (NSCLC) is the most common type of lung cancer, accounting for approximately 85% of all cases. Exemplary non-small cell lung cancers include, but are not limited to, squamous cell (epidermoid) carcinoma, adenocarcinoma, large cell (undifferentiated) carcinoma, adenosquamous carcinoma, and sarcomatoid carcinoma. Thus, in some embodiments, the lung cancer cells are selected from the group consisting of squamous cell carcinoma cells, adenocarcinoma cells, large cell carcinoma cells, adenosquamous carcinoma cells, and sarcomatoid carcinoma cells. Small cell lung cancers (SCLC) comprise about 10-15% of lung cancers. In some embodiments, the cancer cells are small cell lung cancer cells. Lung carcinoid tumors originate in neuroendocrine cells and may be classified by where they form in the lung. Exemplary lung carcinoid tumors include central carcinoids and peripheral carcinoids. Thus, in some embodiments, the cancer cells are central carcinoid tumor cells or peripheral carcinoid tumor cells.

The pancreas is a compound gland that discharges digestive enzymes into the gut (exocrine function) and secretes the hormones insulin and glucagon into the bloodstream (endocrine function). Exemplary pancreatic cancers include, but are not limited to, acinar cell carcinoma, adenocarcinoma (ductal adenocarcinoma), adenosquamous carcinoma, anaplastic carcinoma, cystadenocarcinoma, duct-cell carcinoma (ductal adrenocarcinoma), giant-cell carcinoma (osteoclastoid type), a giant cell tumor, intraductal papillary-mucinous neoplasm (IPMN), mixed-cell carcinoma, mucinous (colloid) carcinoma, mucinous cystadenocarcinoma, papillary adenocarcinoma, pleomorphic giant-cell carcinoma, serous cystadenocarcinoma, small-cell (oat-cell) carcinoma, solid tumors, and pseudopapillary tumors.

In the context of the methods described herein, the term “HR-proficient cancer cells” refers to cancer cells having normal or an increased HR-mediated repair capacity (Kim et al., “ATR-Mediated Proteome Remodeling is a Major Determinant of Homologous Recombination Capacity in Cancer Cells,” Nucleic Acids Research 46(16):8311-8325 (2018), which is hereby incorporated by reference in its entirety). Such cells may have increased HR-mediated repair capacity as a consequence of increased levels of various HR proteins. The increased HR-mediated repair capacity may alleviate oncogene-induced increases in replication stress (Hills et al., “DNA Replication and Oncogene-Induced Replicative Stress,” Current Biology 24:R435-R444 (2014) and Sarni et al., “Oncogene-Induced Replication Stress Drives Genome Instability and Tumorigenesis,” Int. J. Mol. Sci. 18(1339:1-10 (2017), which are hereby incorporated by reference in their entirety). Left unattended, this added replication stress can result in DNA damage or cell death. Consistent with this notion, tolerance to oncogene-induced replication stress has been shown to rely on E2F-dependent transcription and, therefore, ATR signaling to maintain survival (Bertoli et al., “Sustained E2F-Dependent Transcription Is a Key Mechanism to Prevent Replication-Stress-Induced DNA Damage. Cell Rep. 15:1412-1422 (2016), which is hereby incorporated by reference in its entirety).

The term “HR-deficient cancer cells” may refer to cancer cells having one or more mutations in one or more HR proteins (e.g., BRCA1) that results in decreased HR-mediated repair capacity.

The term “sensitize” is a relative term which refers to an increase in the degree of effectiveness of a therapeutic agent (e.g., the PARP inhibitors described herein) in reducing, inhibiting, suppressing growth, or killing of the HR-proficient cancer cells and/or the cancer mediated by HR-proficient cancer cells. The term “growth” as used herein, encompasses any aspect of the growth, proliferation, and progression of cancer cells, including, e.g., viability, cell division (i.e., mitosis), cell growth (e.g., increase in cell size), an increase in genetic material (e.g., prior to cell division), and metastasis. Reduction, inhibition, and/or suppression of HR-proficient cancer cell growth includes, but is not limited to, inhibition of HR-proficient cancer cell growth as compared to the growth of untreated or mock treated cells, reduction in cell viability, inhibition of proliferation, inhibition of metastases, induction of HR-proficient cancer cell senescence, induction of HR-proficient cancer cell death, and reduction of HR-proficient cancer cell size. An increase in sensitivity to a therapy may be measured by, e.g., using cell proliferation assays and/or cell cycle analysis assays.

In some embodiments, the HR-proficient cancer cells and/or the cancer mediated by HR-proficient cancer cells are sensitized to treatment with one or more PARP inhibitors by at least ˜1% (e.g., at least about 1%, at least ˜2%, at least ˜3%, at least ˜4%, at least ˜5%, at least ˜6%, at least ˜7%, at least ˜8%, at least ˜9%, at least ˜10%, at least ˜20%, at least ˜30%, at least ˜40%, at least ˜50%, at least ˜60%, at least ˜70%, at least ˜80%, at least ˜90%, at least ˜95%, at least ˜99%, ˜1%, ˜2%, ˜3%, ˜4%, ˜5%, ˜6%, ˜7%, ˜8%, 9%, ˜10%, ˜20%, ˜30%, ˜40%, ˜50%, ˜60%, ˜70%, ˜80%, ˜90%, ˜95%, ˜99%, ˜100%) as compared to when the HR-proficient cancer cells and/or the cancer mediated by HR-proficient cancer cells are not treated with an ATR inhibitor according to the methods described herein. For example, treatment of HR-proficient cancer cells according to the methods described herein may be effective to decrease the viability or inhibit the proliferation of the HR-proficient cancer cells by at least 10%, at least 20%, at least 30%, at least 40%, at least 50%, at least 60%, at least 70%, at least 80%, or at least 90% following administration of a PARP inhibitor, as compared to when the HR-proficient cancers are not treated with an ATR inhibitor according to the methods described herein.

In some embodiments, the HR-proficient cancer cells and/or the cancer mediated by HR-proficient cancer cells are sensitized to treatment with one or more PARP inhibitors within a range having a lower limit selected from ˜1%, ˜2%, ˜3%, ˜4%, ˜5%, ˜6%, ˜7%, ˜8%, ˜9%, ˜10%, ˜20%, ˜30%, ˜40%, ˜50%, ˜60%, ˜70%, ˜80%, ˜90%, ˜95%, and ˜99%, and an upper limit selected from ˜2%, ˜3%, ˜4%, ˜5%, ˜6%, ˜7%, ˜8%, ˜9%, ˜10%, ˜20%, ˜30%, ˜40%, ˜50%, ˜60%, ˜70%, ˜80%, ˜90%, ˜95%, ˜99%, and ˜100%, or any combination thereof. For example, treatment of HR-proficient cancer cells according to the methods described herein may be effective to decrease the viability or inhibit the proliferation of the HR-proficient cancer cells by 70% to 90% following administration of a PARP inhibitor, as compared to when the HR-proficient cancers are not treated with an ATR inhibitor according to the methods described herein.

In the context of the methods described herein, the HR-proficient cancer cells and/or subject having cancer mediated by HR-proficient cancer cells are treated with an ATR inhibitor. Suitable ATR inhibitors are well known in the art (Gilad et al., “Combining ATR Suppression with Oncogenic Ras Synergistically Increases Genomic Instability, Causing Synthetic Lethality or Tumorigenesis in a Dosage-Dependent Manner,” Cancer Res. 70:9693-9702 (2010); Fokas et al., “Targeting ATR in vivo Using the Novel Inhibitor VE-822 Results in Selective Sensitization of Pancreatic Tumors to Radiation,” Cell Death and Disease 3:1-5 (2012); Min et al., “AZD6738, A Novel Oral Inhibitor of ATR, Induces Synthetic Lethality with ATM Deficiency in Gastric Cancer Cells,” Mol. Cancer. Ther. 16(4):566-577 (2017); and U.S. Pat. No. 8,853,217 B2 to Charrier et al., which are hereby incorporated by reference in their entirety) and include, e.g., VE-822 and AZD6738. Thus, in some embodiments, the ATR inhibitor is VE-822 or AZD6738. Additional suitable ATR inhibitors include, but are not limited to VE-821 and VX-970 (Syljuasen et al., “Targeting Lung Cancer Through Inhibition of Checkpoint Kinases,” Frontiers in Genetics 6 (2015), which is hereby incorporated by reference in its entirety). In some embodiments, the ATR inhibitors are VE-821 or VX-970.

In carrying out the methods of the present application, “treating” or “treatment” includes inhibiting, ameliorating, or delaying onset of a particular condition or state. Treating and treatment also encompasses any improvement in one or more symptoms of the condition or disorder. Treating and treatment encompasses any modification to the condition or course of disease progression as compared to the condition or disease in the absence of therapeutic intervention.

Treating the HR-proficient cancer cell and/or a subject having cancer mediated by HR-proficient cancer cells with the ATR inhibitor may be carried out before, during, after, or in the absence of a PARP inhibitor. For example, in some embodiments, treating the HR-proficient cancer cell and/or a subject having cancer mediated by HR-proficient cancer cells with the ATR inhibitor is carried out daily for at least 5-7 days without administering a PARP inhibitor.

In some embodiments of the methods described herein, treating the HR-proficient cancer cells with an ATR inhibitor is carried out daily with a sub-lethal concentration of ATR. For example, treating the HR-proficient cancer cells with an ATR inhibitor may be carried out daily with a concentration of ATR inhibitor of 0.125 to 0.250 μM (e.g., 0.125 μM, 0.130 μM, 0.135 μM, 0.140 μM, 0.0140 μM, 0.0145 μM, 0.0150 μM, 0.020 μM, or 0.0250 μM). In some embodiments, treating the HR-proficient cancer cells with an ATR inhibitor is carried out daily at a concentration having a lower limit selected from 0.125 to 0.250 μM (e.g., 0.125 μM, 0.130 μM, 0.135 μM, 0.140 μM, 0.0140 μM, 0.0145 μM, 0.0150 μM, and 0.020 μM, and an upper limit selected from 0.130 μM, 0.135 μM, 0.140 μM, 0.0140 μM, 0.0145 μM, 0.0150 μM, 0.020 μM, and 0.0250 μM, and any combination thereof.

In some embodiments of the methods described herein, the ATR inhibitor is VE-821. In accordance with these embodiments, treating the HR-proficient cancer cells with VE-821 is carried out daily with a concentration of ATR inhibitor of 2.5 to 5.0 μM (e.g., 2.5 μM, 2.6 μM, 2.7 μM, 2.8 μM, 2.9 μM, 3.0 μM, 3.1 μM, 3.2 μM, 3.3 μM, 3.4 μM, 3.5 μM, 3.6 μM, 3.7 μM, 3.8 μM, 3.9 μM, 4.0 μM, 4.1 μM, 4.2 μM, 4.3 μM, 4.4 μM, 4.5 μM, 4.6 μM, 4.7 μM, 4.8 μM, 4.9 μM, or 5.0 μM). In some embodiments, treating the HR-proficient cancer cells with VE-821 is carried out daily at a concentration having a lower limit selected from 2.5 μM, 2.6 μM, 2.7 μM, 2.8 μM, 2.9 μM, 3.0 μM, 3.1 μM, 3.2 μM, 3.3 μM, 3.4 μM, 3.5 μM, 3.6 μM, 3.7 μM, 3.8 μM, 3.9 μM, 4.0 μM, 4.1 μM, 4.2 μM, 4.3 μM, 4.4 μM, 4.5 μM, 4.6 μM, 4.7 μM, 4.8 μM, and 4.9 μM, and an upper limit selected from 2.6 μM, 2.7 μM, 2.8 μM, 2.9 μM, 3.0 μM, 3.1 μM, 3.2 μM, 3.3 μM, 3.4 μM, 3.5 μM, 3.6 μM, 3.7 μM, 3.8 μM, 3.9 μM, 4.0 μM, 4.1 μM, 4.2 μM, 4.3 μM, 4.4 μM, 4.5 μM, 4.6 μM, 4.7 μM, 4.8 μM, 4.9 μM, and 5.0 μM, and any combination thereof.

The methods of sensitizing HR-proficient cancer cells to treatment with a PARP inhibitor described herein may be carried out in vitro, in vivo, or ex vivo. When methods described herein are carried out in vivo, selecting HR-proficient cancer cells may involve selecting a subject as described herein and administering the PARP inhibitor as described herein to the selected subject/tumor.

Suitable subjects in accordance with the methods described herein include, without limitation, mammals. In some embodiments, the subject is selected from the group consisting of primates (e.g., humans, monkeys), equines (e.g., horses), bovines (e.g., cattle), porcines (e.g., pigs), ovines (e.g., sheep), caprines (e.g., goats), camelids (e.g., llamas, alpacas, camels), rodents (e.g., mice, rats, guinea pigs, hamsters), canines (e.g., dogs), felines (e.g., cats), leporids (e.g., rabbits). In some embodiments, the selected subject is an agricultural animal, a domestic animal, or a laboratory animal. In some embodiments, the subject is a human subject. Suitable human subjects include, without limitation, infants, children, adults, and elderly subjects.

In some embodiments, the subject is one who has been diagnosed with a HR-proficient cancer or the cancer is mediated by HR-proficient cancer cells.

In some embodiments of the methods described herein, treating the selected subject is carried out with a sub-lethal concentration of ATR. For example, treating the selected subject with an ATR inhibitor may be carried out daily with a concentration of ATR inhibitor of 0.5 to 4.0 mg per the subject's mass in kilograms (e.g., 0.5, 0.6, 0.7. 0, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mg per the subject's mass in kilograms). In some embodiments, treating the selected subject with an ATR inhibitor is carried out daily at a concentration having a lower limit selected from 0.5, 0.6, 0.7. 0, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, and 3.9 mg per the subject's mass in kilograms, and an upper limit selected from 0.6, 0.7. 0, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, and 4.0 mg per the subject's mass in kilograms, and any combination thereof.

In some embodiments of the methods described herein, the ATR inhibitor is VE-821. In accordance with these embodiments, treating the selected subject with VE-821 is carried out daily with a concentration of ATR inhibitor of 5.0 to 80.0 milligrams per the subject's mass in kilograms (e.g., 5.0, 10.0, 15.0, 20.0, 25.0, 30.0, 35.0, 40.0, 45.0, 50.0, 55.0, 60.0, 65.0, 70.0, 75.0, or 80.0 milligrams per the subject's mass in kilograms). In some embodiments, treating the selected subject with VE-821 is carried out daily at a concentration having a lower limit selected from 5.0, 10.0, 15.0, 20.0, 25.0, 30.0, 35.0, 40.0, 45.0, 50.0, 55.0, 60.0, 65.0, 70.0, and 75.0 milligrams per the subject's mass in kilograms, and an upper limit selected from 10.0, 15.0, 20.0, 25.0, 30.0, 35.0, 40.0, 45.0, 50.0, 55.0, 60.0, 65.0, 70.0, 75.0, and 80.0 milligrams per the subject's mass in kilograms, and any combination thereof.

As described herein, treating HR-proficient cancer cells and/or a subject having a cancer mediated by HR-proficient cancer cells according to the methods described herein is effective to reduce the abundance of HR proteins include, e.g., BRCA1, FANCJ, RRM2, CTIP, RAD51, and/or TOPBP1, as compared to when HR-proficient cancer cells are untreated. Applicants have surprisingly found that the reduction in the abundance of HR proteins resulting from chronic inhibition of ATR (e.g., sub-lethal daily administration of an ATR inhibitor for at least 5-7 days in the absence of a PARP inhibitor) is sufficient to sensitize HR-proficient cancer cells to treatment with a PARP inhibitor as compared to when the HR-proficient cancer cells and/or the cancer mediated by HR-proficient cancer cells are not treated with an ATR inhibitor according to the methods described herein. Thus, in some embodiments, sensitizing converts HR-proficient cancer cells to HR-deficient cancer cells.

A further aspect of the present application relates to a method of treating HR-proficient cancer cells. This method involves providing HR-proficient cancer cells; treating the HR-proficient cancer cells with an ATR inhibitor, where the treating is carried out without administering PARP inhibitors; and administering to the treated HR-proficient cells an ATR inhibitor and a PARP inhibitor.

Yet another aspect of the present application relates to a method of treating a cancer patient. This method involves selecting a subject with a cancer mediated by HR-proficient cells; treating the selected subject with an ATR inhibitor, where the treating is carried out without administering PARP inhibitors; and administering to the selected subject an ATR inhibitor and a PARP inhibitor.

As described herein, treating the HR-proficient cancer cells and/or subject having a cancer mediated by HR-proficient cancer cells with an ATR inhibitor is effective to confer a synthetic lethality to HR-proficient cancer cells when a PARP inhibitor is subsequently administered. Thus, in some embodiments, the treating is carried out under conditions insufficient to kill the HR-proficient cancer cells and where the administering is carried out under conditions sufficient to kill cancer cells but insufficient to kill non-cancer cells.

The treating may be carried out as described above.

Poly (ADP-ribose) polymerase (PARP) is an enzyme involved in repairing DNA single-strand breaks (SSBs) via base excision repair (Hoeijmakers, J H, “Genome Maintenance Mechanisms for Preventing Cancer,” Nature 411(6835):366-374 (2001), which is hereby incorporated by reference in its entirety). Once activated by damaged DNA fragments, e.g., after exposure to chemotherapy, ionizing radiation, oxygen free radicals, or nitric oxide (NO), PARP catalyzes the transfer of ADP-ribose units from nicotinamide adenine dinucleotide (NAD⁺) to nuclear acceptor proteins, and is responsible for the formation of protein-bound linear and branched homo-ADP-ribose polymers. PARP activation results in the attachment of up to 100 ADP-ribose units to a variety of nuclear proteins, including histones, topoisomerases, DNA and RNA polymerases, DNA ligases, Ca²⁺- and Mg²⁺-dependent endonucleases, and PARP itself. Thus, PARP plays a role in enhancing DNA repair and maintaining DNA integrity.

In the presence of PARP inhibitors, unrepaired SSBs may develop into double strand breaks (DSBs) that require HR to repair properly. PARP inhibitors have also been proposed to trap PARP-1 on to DNA repair intermediates, leading to obstruction of replication forks and further induction of DSBs (Helleday, T., “The Underlying Mechanism for the PARP and BRCA Synthetic Lethality: Clearing up the Misunderstandings,” Mol. Oncol. 5(4):387-393 (2011), which is hereby incorporated by reference in its entirety). Therefore, PARP inhibitors are highly effective for treating BRCA-deficient cancers, as they confer synthetic lethality to cells with defective HR-mediated repair resulting from BRCA1 or BRCA2 mutations (Bryant et al., “Specific Killing of BRCA2-Deficient Tumours with Inhibitors of Poly(ADP)-Ribose Polymerase,” Nature 434(7035):913-917 (2005); Farmer et al., “Targeting DNA Repair Defect in BRCA Mutant Cells as a Therapeutic Strategy,” Nature 434:917*921 (2005); Rottenberg et al., “High Sensitivity of BRCA1-Deficient Mammary Tumors to the PARP Inhibitor AZD2281 Alone and in Combination with Platinum Drugs,” PNAS 105(44):17079-17084 (2008); and Fong et al., “Inhibition of Poly(ADP-Ribose) Polymerase in Tumors from BRCA Mutation Carriers,” N. Engl. J. Med. 361(2):123-34 (2009), which are hereby incorporated by reference in their entirety). Despite the effectiveness of PARP inhibitors, some BRCA-deficient cancers have been shown to develop resistance to PARP inhibitor treatment (Fojo et al., “Mechanisms of Resistance to PARP Inhibitors—Three and Counting,” Cancer Discov. 3(1):20-23 (2013); Lord et al., “Mechanisms of Resistance to Therapies Targeting BRCA-Mutant Cancers,” Nat. Med. 19(11):1381-1388 (2013); and Sonnenblick et al., “An Update on PARP Inhibitors—Moving to the Adjuvant Setting,” Nat. Rev. Clin. Oncol. 12(1):27-41 (2015), each of which are hereby incorporated by reference in their entirety). Among the multiple mechanisms of resistance that have been discovered, one common mechanism involves a rewiring of the HR pathway to bypass the need for BRCA1 or BRCA2. For example, deletion of the anti-resection factor 53BP1 has been shown to restore HR-mediated repair in BRCA1-deficient cells, thus conferring PARP inhibitor resistance (Bunting et al., “53BPI Inhibits Homologous Recombination in Brca1-Deficient Cells by Blocking Resection of DNA Breaks,” Cell 141(2):243-254 (2010) and Jaspers et al., “Loss of 53BPI Causes PARP Inhibitor Resistance in Brca1—Mutated Mouse Mammary Tumors,” Cancer Discov. 3(1):68-81 (2013), which are hereby incorporated by reference in their entirety). Co-treatment with ATR inhibitors and PARP inhibitors has been proposed to prevent the emergence of PARP inhibitor resistance altogether (Yazinski et al., “ATR Inhibition Disrupts Rewired Homologous Recombination Fork Protection Pathways in PARP Inhibitor-Resistant BRCA-Deficient Cancer Cells,” Genes Dev. 31(3):318-332 (2017), which is hereby incorporated by reference in its entirety).

Applicants have surprisingly found that ATR controls the abundance of HR proteins (e.g., BRCA1, FANCJ, RRM2, CTIP, RAD51, and/or TOPBP1) through the control of transcription and, in the case of BRCA1, also through the control of protein stability. Thus, in the context of the methods described herein, continuous low-dose (sub-lethal) ATR inhibition of HR-proficient cancer cells depletes HR factors (i.e., results in an HR-deficiency) and confers a synthetic lethality when such cells are subsequently administered a PARP inhibitor.

PARP inhibitors are well known in the art (Walsh C., “Targeted Therapy for Ovarian Cancer: The Rapidly Evolving Landscape of PARP Inhibitor Use,” Minerva Ginecol. 70(2):150-170 (2018); McLachlan et al., “The Current Status of PARP Inhibitors in Ovarian Cancer,” Tumori. 102(5):433-440 (2016); Sargazi et al., “Novel Poly (Adenosine Diphosphate-Ribose) Polymerase (PARP) Inhibitor, AZD2461, Down-Regulates VEGF and Induces Apoptosis in Prostate Cancer,” Iran Biomed. J. (2019); and U.S. Pat. No. 9,150,628 B2 to Hamiche, which are hereby incorporated by reference in their entirety).

Suitable PARP inhibitors for use in the methods described herein include, without limitation, olaparib (AZD 2281), rucaparib (AG 014699), niraparib (MK 4827), talozaparib (BMN 673), AZD 2461, and veliparib (ABT-888). Additional suitable PARP inhibitors include, without limitation, iniparib (BSI-201), CEP-9722, INO-1001, MK-4827, E7016, and BMN673 (see, e.g., U.S. patent application Ser. No. 14/816,432 to Pollard et al., which is hereby incorporated by reference in its entirety).

In some embodiments, the PARP inhibitor is administered to the treated HR-proficient cancer cells daily at a concentration of 2.5 to 10 μM (e.g., 2.5 μM, 2.6 μM, 2.7 μM, 2.8 μM, 2.9 μM, 3.0 μM, 3.1 μM, 3.2 μM, 3.3 μM, 3.4 μM, 3.5 μM, 3.6 μM, 3.7 μM, 3.8 μM, 3.9 μM, 4.0 μM, 4.1 μM, 4.2 μM, 4.3 μM, 4.4 μM, 4.5 μM, 4.6, 4.7 μM, 4.8 μM, 4.9 μM, 5.0 μM, 5.1 μM, 5.2 μM, 5.3 μM, 5.4 μM, 5.5 μM, 5.6 μM, 5.7 μM, 5.8 μM, 5.9 μM, 6.0 μM, 6.1 μM, 6.2 μM, 6.3 μM, 6.4 μM, 6.5 μM, 6.6 μM, 6.7 μM, 6.8 μM, 6.9 μM, 7.0 μM, 7.1 μM, 7.2 μM, 7.3 μM, 7.4 μM, 7.5 μM, 7.6 μM, 7.7 μM, 7.8 μM, 7.9 μM, 8.0 μM, 8.1 μM, 8.2 μM, 8.3 μM, 8.4 μM, 8.5 μM, 8.6 μM, 8.7 μM, 8.8 μM, 8.9 μM, 9.0 μM, 9.1 μM, 9.2 μM, 9.3 μM, 9.4 μM, 9.5 μM, 9.6 μM, 9.7 μM, 9.8 μM, 9.9 μM, or 10.0 μM). In some embodiments, the PARP inhibitor is administered to the treated HR-proficient cancer cells daily at a concentration having a lower limit selected from 2.5 μM, 2.6 μM, 2.7 μM, 2.8 μM, 2.9 μM, 3.0 μM, 3.1 μM, 3.2 μM, 3.3 μM, 3.4 μM, 3.5 μM, 3.6 μM, 3.7 μM, 3.8 μM, 3.9 μM, 4.0 μM, 4.1 μM, 4.2 μM, 4.3 μM, 4.4 μM, 4.5 μM, 4.6, 4.7 μM, 4.8 μM, 4.9 μM, 5.0 μM, 5.1 μM, 5.2 μM, 5.3 μM, 5.4 μM, 5.5 μM, 5.6 μM, 5.7 μM, 5.8 μM, 5.9 μM, 6.0 μM, 6.1 μM, 6.2 μM, 6.3 μM, 6.4 μM, 6.5 μM, 6.6 μM, 6.7 μM, 6.8 μM, 6.9 μM, 7.0 μM, 7.1 μM, 7.2 μM, 7.3 μM, 7.4 μM, 7.5 μM, 7.6 μM, 7.7 μM, 7.8 μM, 7.9 μM, 8.0 μM, 8.1 μM, 8.2 μM, 8.3 μM, 8.4 μM, 8.5 μM, 8.6 μM, 8.7 μM, 8.8 μM, 8.9 μM, 9.0 μM, 9.1 μM, 9.2 μM, 9.3 μM, 9.4 μM, 9.5 μM, 9.6 μM, 9.7 μM, 9.8 μM, and 9.9 μM, and an upper limit selected from 2.6 μM, 2.7 μM, 2.8 μM, 2.9 μM, 3.0 μM, 3.1 μM, 3.2 μM, 3.3 μM, 3.4 μM, 3.5 μM, 3.6 μM, 3.7 μM, 3.8 μM, 3.9 μM, 4.0 μM, 4.1 μM, 4.2 μM, 4.3 μM, 4.4 μM, 4.5 μM, 4.6, 4.7 μM, 4.8 μM, 4.9 μM, 5.0 μM, 5.1 μM, 5.2 μM, 5.3 μM, 5.4 μM, 5.5 μM, 5.6 μM, 5.7 μM, 5.8 μM, 5.9 μM, 6.0 μM, 6.1 μM, 6.2 μM, 6.3 μM, 6.4 μM, 6.5 μM, 6.6 μM, 6.7 μM, 6.8 μM, 6.9 μM, 7.0 μM, 7.1 μM, 7.2 μM, 7.3 μM, 7.4 μM, 7.5 μM, 7.6 μM, 7.7 μM, 7.8 μM, 7.9 μM, 8.0 μM, 8.1 μM, 8.2 μM, 8.3 μM, 8.4 μM, 8.5 μM, 8.6 μM, 8.7 μM, 8.8 μM, 8.9 μM, 9.0 μM, 9.1 μM, 9.2 μM, 9.3 μM, 9.4 μM, 9.5 μM, 9.6 μM, 9.7 μM, 9.8 μM, 9.9 μM, and 10.0, and any combination thereof.

In some embodiments, the PARP inhibitor is administered to the selected subject daily at a concentration of 2.5 to 10 grams per the subject's mass in kilograms (e.g., 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 grams per the subject's mass in kilograms). In some embodiments, the PARP inhibitor is administered to the selected subject daily at a concentration having a lower limit selected from 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, and 9.9 grams per the subject's mass in kilograms, and an upper limit selected from 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0 grams per the subject's mass in kilograms, and any combination thereof.

In all aspects of the present application that involve administering combination(s) of therapeutic agents (e.g., the ATR inhibitor and/or the PARP inhibitor described herein), the combination of therapeutic agents may be administered about 1 day after, about 2 days after, about 3 days after, about 4 days after, about 5 days after, about 6 days after, or about 1 week after the treating is carried out.

In some embodiments, the treating is carried out daily for at least 5-7 days and the administering is carried out daily for at least 2-3 days.

Suitable ATR inhibitors and concentrations for carrying out the methods described herein are described in detail above.

Suitable cancers and cancer cells are described in detail above.

As used herein, “synthetic lethal” or “synthetic lethality” refers to the condition that arises when a combination of deficiencies in the function of two or more gene products leads to cell death, whereas a deficiency in only one of these gene products does not. The deficiencies can arise through mutations, epigenetic alterations, or inhibitors of one of the genes or gene products. In some embodiments, the methods described herein are synthetic lethal to HR-proficient cancer cells when the treating with an ATR inhibitor is carried out without administering PARP inhibitors and the administering is carried out with the combination of an ATR inhibitor and a PARP inhibitor.

The therapeutic agents and combinations for use in the methods described herein can be formulated according to any available conventional method. Examples of preferred dosage forms include a tablet, a powder, a subtle granule, a granule, a coated tablet, a capsule, a syrup, a troche, an inhalant, a suppository, an injectable, an ointment, an ophthalmic ointment, an eye drop, a nasal drop, an ear drop, a cataplasm, a lotion and the like. In the formulation, generally used additives such as a diluent, a binder, an disintegrant, a lubricant, a colorant, a flavoring agent, and if necessary, a stabilizer, an emulsifier, an absorption enhancer, a surfactant, a pH adjuster, an antiseptic, an antioxidant and the like can be used. In addition, the formulation is also carried out by combining compositions that are generally used as a raw material for pharmaceutical formulation, according to conventional methods. Examples of these compositions include, for example, (1) an oil such as a soybean oil, a beef tallow and synthetic glyceride; (2) hydrocarbon such as liquid paraffin, squalane and solid paraffin; (3) ester oil such as octyldodecyl myristic acid and isopropyl myristic acid; (4) higher alcohol such as cetostearyl alcohol and behenyl alcohol; (5) a silicon resin; (6) a silicon oil; (7) a surfactant such as polyoxyethylene fatty acid ester, sorbitan fatty acid ester, glycerin fatty acid ester, polyoxyethylene sorbitan fatty acid ester, a solid polyoxyethylene castor oil and polyoxyethylene polyoxypropylene block co-polymer; (8) water soluble macromolecule such as hydroxyethyl cellulose, polyacrylic acid, carboxyvinyl polymer, polyethyleneglycol, polyvinylpyrrolidone and methylcellulose; (9) lower alcohol such as ethanol and isopropanol; (10) multivalent alcohol such as glycerin, propyleneglycol, dipropyleneglycol and sorbitol; (11) a sugar such as glucose and cane sugar; (12) an inorganic powder such as anhydrous silicic acid, aluminum magnesium silicicate and aluminum silicate; (13) purified water, and the like.

Additives for use in the above formulations may include, for example, (1) lactose, corn starch, sucrose, glucose, mannitol, sorbitol, crystalline cellulose and silicon dioxide as the diluent; (2) polyvinyl alcohol, polyvinyl ether, methyl cellulose, ethyl cellulose, gum arabic, tragacanth, gelatine, shellac, hydroxypropyl cellulose, hydroxypropylmethyl cellulose, polyvinylpyrrolidone, polypropylene glycol-poly oxyethylene-block co-polymer, meglumine, calcium citrate, dextrin, pectin and the like as the binder; (3) starch, agar, gelatine powder, crystalline cellulose, calcium carbonate, sodium bicarbonate, calcium citrate, dextrin, pectic, carboxymethylcellulose/calcium and the like as the disintegrant; (4) magnesium stearate, talc, polyethyleneglycol, silica, condensed plant oil and the like as the lubricant; (5) any colorants whose addition is pharmaceutically acceptable is adequate as the colorant; (6) cocoa powder, menthol, aromatizer, peppermint oil, cinnamon powder as the flavoring agent; (7) antioxidants whose addition is pharmaceutically accepted such as ascorbic acid or alpha-tophenol.

The therapeutic agents and combinations for use in the methods described herein can be formulated into a pharmaceutical composition as any one or more of the active compounds described herein and a physiologically acceptable carrier (also referred to as a pharmaceutically acceptable carrier or solution or diluent). Such carriers and solutions include pharmaceutically acceptable salts and solvates of compounds used in the methods described herein, and mixtures comprising two or more of such compounds, pharmaceutically acceptable salts of the compounds and pharmaceutically acceptable solvates of the compounds. Such compositions are prepared in accordance with acceptable pharmaceutical procedures such as described in Remington: The Science and Practice of Pharmacy, 20th edition, ed. Alfonso R. Gennaro (2000), which is hereby incorporated by reference in its entirety.

The term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the therapeutic agent.

Reference to therapeutic agents described herein (i.e., the ATR inhibitor and/or the PARP inhibitor) includes any analog, derivative, isomer, metabolite, pharmaceutically acceptable salt, pharmaceutical product, hydrate, N-oxide, crystal, polymorph, prodrug, or any combination thereof.

The therapeutic agents in a free form can be converted into a salt, if need be, by conventional methods. The term “salt” used herein is not limited as long as the salt is pharmacologically acceptable; preferred examples of salts include a hydrohalide salt (for instance, hydrochloride, hydrobromide, hydroiodide and the like), an inorganic acid salt (for instance, sulfate, nitrate, perchlorate, phosphate, carbonate, bicarbonate and the like), an organic carboxylate salt (for instance, acetate salt, maleate salt, tartrate salt, fumarate salt, citrate salt and the like), an organic sulfonate salt (for instance, methanesulfonate salt, ethanesulfonate salt, benzenesulfonate salt, toluenesulfonate salt, camphorsulfonate salt and the like), an amino acid salt (for instance, aspartate salt, glutamate salt and the like), a quaternary ammonium salt, an alkaline metal salt (for instance, sodium salt, potassium salt and the like), an alkaline earth metal salt (magnesium salt, calcium salt and the like) and the like. In addition, hydrochloride salt, sulfate salt, methanesulfonate salt, acetate salt and the like are preferred as “pharmacologically acceptable salt” of the compounds disclosed herein.

In certain embodiments, the therapeutic agents disclosed herein may be in a prodrug form, meaning that it must undergo some alteration (e.g., oxidation or hydrolysis) to achieve its active form.

The present application may be further illustrated by reference to the following examples.

### EXAMPLES

The following examples are provided to illustrate embodiments of the present invention but they are by no means intended to limit its scope.

### Materials and Methods for Examples 1-6

### Mammalian Cell Culture

Human U2OS, HCT116, hTERT RPE-1, HEK293T, HeLa, U87, T98G, A2780, and human skin fibroblast cells (GM08398 and GM18366) were grown in DMEM media supplemented with 10% bovine calf serum, penicillin/streptomycin and non-essential amino acids. SV40 large T antigen transformed hTERT RPE-1 cells were generated by infecting hTERT RPE-1 cells with a SV40 large T antigen (SV40 LT)-expressing retrovirus and were selected using 500 μg/ml G418. The stable SV40 large T antigen transformed hTERT RPE-1 cell line was then maintained in culture media supplemented 500 μg/ml G418. For chronic treatment with ATR inhibitor (ATRi, VE-821) or ATM inhibitor (ATMi, KU-55933), cells were maintained in media with 5 μM ATR or ATM inhibitor respectively for 8 days before cells were subjected to western blot analysis, proteomic analysis, DR-GFP/EJ5-GFP assay or immunofluorescent staining. For CHK1 inhibitor (UCN-01), cells were treated for 5 days with 0.2 μM of inhibitor before the analysis. Primary human fibroblast cells from control subjects (GM08398) and patients with Seckel syndrome (GM18366, ATR-defective) were obtained from Coriell Institute for Medical Research, Camden, N.J.

### DR-GFP and EJ5-GFP Assays

For the DR-GFP assay, cells (U2OS, HCT116, hTERT RPE-1, HEK293T, HeLa, U87, T98G, A2780, 293-T-REX-E2F6 cells and SV40 large T antigen transformed hTERT RPE-1) were transfected with the mCherry plasmid or DR-GFP reporter plasmid (pDR-GFP; addgene plasmid 26475) together with a plasmid coding for I-SceI (pCBASceI; addgene plasmid 26477) or DR-GFP reporter plasmid together with an empty plasmid pCAGGS. Two days after transfection, cells were trypsinized, resuspended in PBS and then analyzed by flow cytometry using FACSAria Fusion or Accuri C6 cytometer (BD). In each experiment, the percentage of GFP-positive cells from the sample transfected with empty plasmid pCAGGS was subtracted from the percentage of GFP-positive cells in the sample transfected with I-SceI. The GFP percentage was then normalized by the mCherry transfection efficiency. Each graph was plotted based on at least 3 independent experiments showing mean±SEM (N≥3). For the EJ5-GFP assay, EJ5-GFP reporter plasmid (pimEJ5GFP, addgene plasmid 44026) was used instead of DR-GFP reporter plasmid and the assay was performed as described for DR-GFP assay.

### Immunofluorescence

Primary human fibroblast cells were grown on glass coverslips and irradiated at 10 Gy. Cells were then fixed with 3.7% formaldehyde/PBS for 15 minutes at room temperature 8 hours post-irradiation. Fixed cells were then washed with PBS, permeabilized with 0.2% Triton X-100 for 5 minutes at room temperature and blocked with 5% BSA at 37° C. for 30 minutes. Blocked samples were incubated with primary antibodies for 1 hour at room temperature, followed by three PBS washes and secondary antibody incubation using Alexa Fluor488 donkey anti-rabbit (A-21206; Thermo Fisher Scientific) for 1 hour. After secondary antibody incubation, cells were washed in PBS for three times and mounted onto glass microscope slides using Vectashield mounting media (H1200; Vector Laboratory).

### Microscopy Analysis

Fixed cells were imaged at room temperature using a CSU-X spinning disc confocal microscope (Intelligent Imaging Innovations) on an inverted microscope (DMI600B; Leica), with 63×1.4 NA objective lens and a charge-coupled device camera (cool-SNAP HQ2, Photometrics). Z stack images were obtained and saved in Slidebook software (Intelligent, Imaging, Innovations), where maximum intensity projections were created. For RAD51 and BRCA1 foci analysis in control skin fibroblast and Seckel cells, more than 150 cells for each sample were imaged and analyzed per replicate. The fraction of cells with more than 10 distinct RAD51 foci or more than 5 BRCA1 foci were determined for each replicate. The graph was plotted using the arithmetic mean and SEM derived from 4 independent biological replicates. A two-tailed Student's t test with 95% confidence interval was used for the statistical analysis.

### Immunoblotting Analysis

Cells were harvested and lysed in modified radioimmunoprecipitation assay (RIPA) buffer (50 mM Tris-HCl pH 7.5, 150 mM NaCl, 1% tergitol, 0.25% sodium deoxycholate, 5 mM EDTA) supplemented with Complete EDTA-free protease inhibitor cocktail (Roche), 1 mM PMSF, 5 mM sodium fluoride, 10 mM β-glycerol-phosphate and 0.4 mM sodium orthovanadate. Whole cell lysates were cleared with 5 minute centrifugation at 13,000 rpm at 4° C. and mixed with 3×SDS sample buffer (bromophenol blue, stacking gel buffer, 50% glycerol, 3% SDS and 60 mM DTT). After being resolved on SDS-PAGE gels, proteins were then transferred onto polyvinylidene difluoride (PVDF) membranes and detected with designated antibodies.

### Proteomic Analysis

For quantitative proteomic analysis, U2OS cells chronically treated with DMSO or ATR inhibitor were cultured, respectively, in “light” or “heavy” SILAC DMEM media (ThermoFisher Scientific 88425) supplemented with 10% dialyzed FBS and penicillin/streptomycin. “Light” SILAC media was supplemented with “light” (normal) arginine ¹²C₆, ¹⁴N₂ and lysine ¹²C₆, ¹⁴N₄, while “heavy” SILAC media contained “heavy” lysine ¹³C₆, ¹⁵N₂ and “heavy” arginine ¹³C₆, ¹⁵N₄. From SILAC cells chronically treated with DMSO or ATR inhibitor, nuclei were isolated using hypotonic buffer and further lysed in modified RIPA buffer. The nuclear lysates were cleared by centrifugation for 5 minutes at 4° C. The nuclear proteins were reduced, alkylated, precipitated and trypsin-digested. The peptides were than desalted, dried, and resuspended in 80% acetonitrile and 1% formic acid and then fractionated using Hydrophilic Interaction Chromatography (HILIC). HILIC fractions were dried and reconstituted in 0.1% trifluoroacetic acid and analyzed using a Q-Exactive Orbitrap and Lumos mass spectrometer. Database search and quantitation of heavy/light peptide isotope ratios were performed using Sorcerer as previously described (Bastos de Oliveira et al., “Phosphoproteomics Reveals Distinct Modes of Mec1/ATR Signaling During DNA Replication,” Mol. Cell 57:1124-1132 (2015) and Bastos de Oliveira et al., “Quantitative Analysis of DNA Damage Signaling Responses to Chemical and Genetic Perturbations,” Methods Mol. Biol. 1672:645-660 (2018), which are hereby incorporated by reference in their entirety). The mass spectrometry proteomics data have been deposited to the ProteomeXchange Consortium via the PRIDE (Proteomics Identifications) (Vizcaino et al., “2016 update of the PRIDE database and its related tools,” Nucleic Acids Res. 44:11033 (2016), which is hereby incorporated by reference in its entirety) partner repository with the data set identifier PXD010223.

### Chemicals and Antibodies

The following chemicals were used in the study: ATR inhibitor (VE-821) and ATM inhibitor (KU-55933) from Selleckchem, CHK1 inhibitor (UCN-01) from Sigma, Geneticin (G418 sulfate) from Thermo Fisher Scientific, and Cycloheximide (CHX) from MP Bio. The following antibodies were used in western blot analysis and immunofluorescent staining: anti-53BP1 and anti-FANCJ from Novus Biologicals, anti-phospho-CHK1 (Ser345) from Cell Signaling, anti-RAD51 from Millipore, anti-CHK1, anti-E2F6 and anti-E2F1 from Santa Cruz Biotechnology, anti-Actin and anti-Tubulin from Sigma, ECL HRP-linked secondary antibody from GE healthcare. Rabbit polyclonal anti-TOPBP1 and anti-BRCA1 antibodies were home-raised as previously described (Danielsen et al., “HCLK2 Is Required for Activity of the DNA Damage Response Kinase ATR,” J. Biol. Chem. 284:4140-4147 (2009) and Kakarougkas et al., “Co-operation of BRCA1 and POH1 Relieves the Barriers Posed by 53BP1 and RAP80 to Resection,” Nucleic Acids Res. 41:10298-10311 (2013), which are hereby incorporated by reference in their entirety). Anti-RRM2 serum was raised in rabbit against an immunogen containing the first 250 amino acids of human RRM2.

### Cell Cycle Analysis

Cells were harvested by standard trypsinization and fixed with 70% ethanol. After fixation, cells from each sample were resuspended in PBS, permeabilized using 0.1% Triton X-100 and treated with RNase A (0.8 mg/ml). Then cells were stained with propidium iodide and analyzed by flow cytometry using FACSAria Fusion (BD) or Accuri C6 cytometer (BD) to determine the fraction of cells in different stages of the cell cycle.

### Real Time PCR

Cells cultured from 10 cm dish were collected and lysed in 1 ml of TRIzol (Invitrogen). Chloroform was added to the lysed sample to separate RNA. Separated RNA was precipitated by isopropyl alcohol, followed by two washes with 75% ethanol. 1 μg of extracted RNA from each sample was then reverse transcribed with Bio-Rad iScript cDNA synthesis kit. The samples (˜100 ng) were analyzed in a Roche 480 light cycler in a total of 15 μl containing SYBR green mix and primers. The values were calculated using “absolute quantification/2^{nd} derivative max” in lightcycler 480 program and then normalized to the level of GAPDH. Primers used in the study are shown in Table 1 below.

TABLE 1
Primers

| Name | Sequence | SEQ ID NO: |
|---|---|---|
| BRCA1-F | ACCTTGGAACTGTGAGAACTCT | SEQ ID NO: 1 |
| BRCA1-R | TCTTGATCTCCCACACTGCAATA | SEQ ID NO: 2 |
| RAD51-F | CAACCCATTTCACGGTTAGAGC | SEQ ID NO: 3 |
| RAD51-R | TTCTTTGGCGCATAGGCAACA | SEQ ID NO: 4 |
| 53BP1-F | ATGGACCCTACTGGAAGTCAG | SEQ ID NO: 5 |
| 53BP1-R | TTTCTTTGTGCGTCTGGAGATT | SEQ ID NO: 6 |
| GAPDH-F | TCATTGACCTCAACTACATGGTTT | SEQ ID NO: 7 |
| GAPDH-R | GGAAGATGGTGATGGGATTTC | SEQ ID NO: 8 |

### Crystal Violet Staining Survival Assay

First, hTERT RPE-1, U2OS, HCT116, HeLa, A2780, or U87 cells were treated for 5 days with either DMSO or 5 μM ATR inhibitor. Next, 1×10⁵ cells of each cell type were plated onto a 10 cm dish and further treated with DMSO, ATR inhibitor, and/or PARP inhibitor for an additional 3 days. Cells were then washed with PBS and either fixed in 100% methanol for 15 minutes at 4° C. or fixed after additional 2 or 8 days of culture in drug-free media. Fixed plates were stained with 0.1% crystal violet solution overnight followed by a distilled water wash. Pictures were taken after the plates were dried. For quantitation of cell viability, total viable cells were counted using a hematocytometer after indicated treatment.

### Example 1—Chronic ATR Inhibition Severely Impairs HR-Mediated Repair

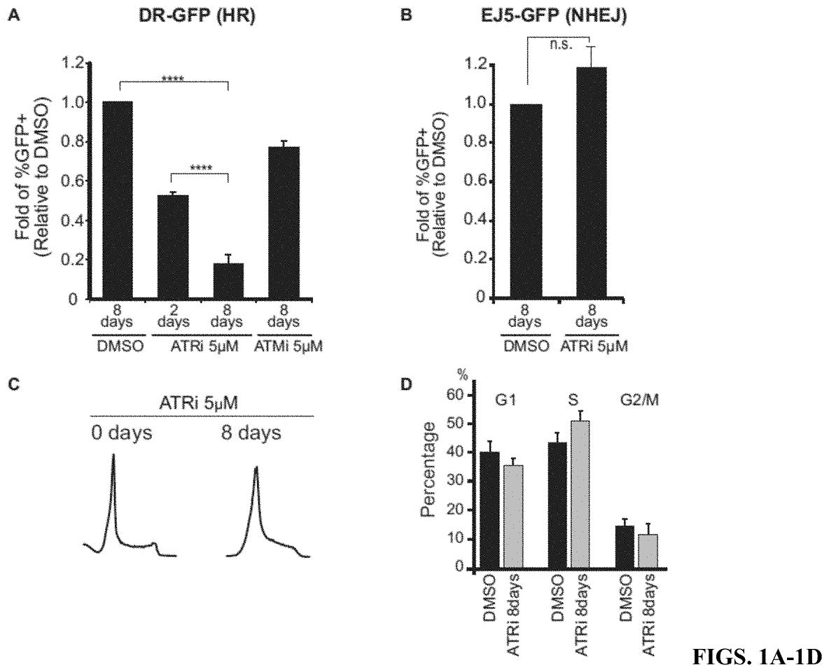

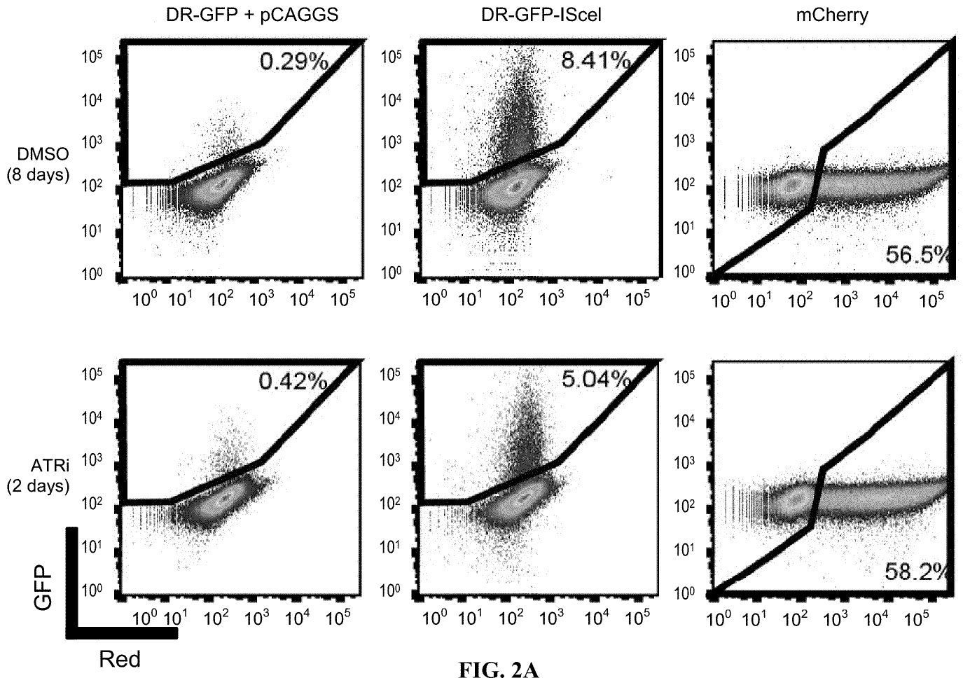

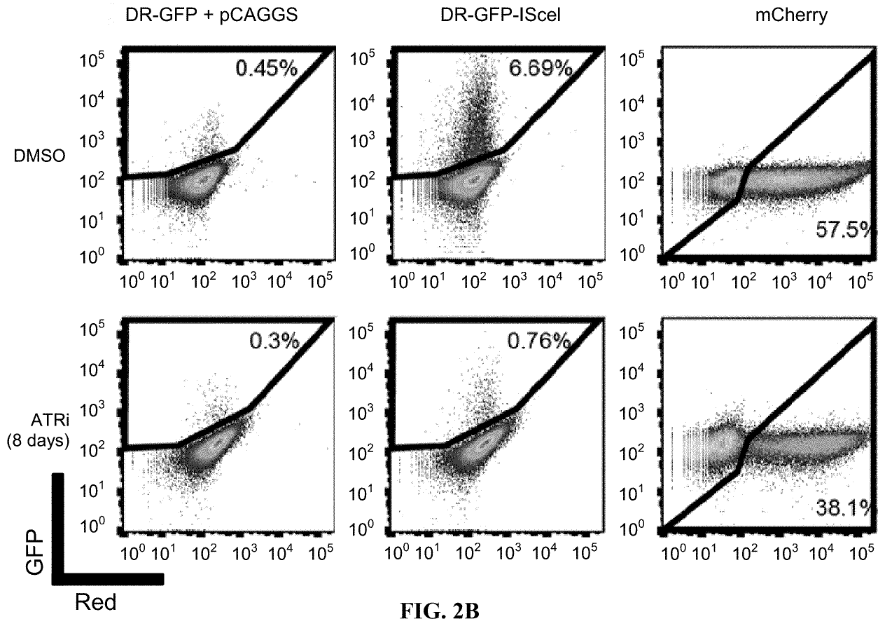

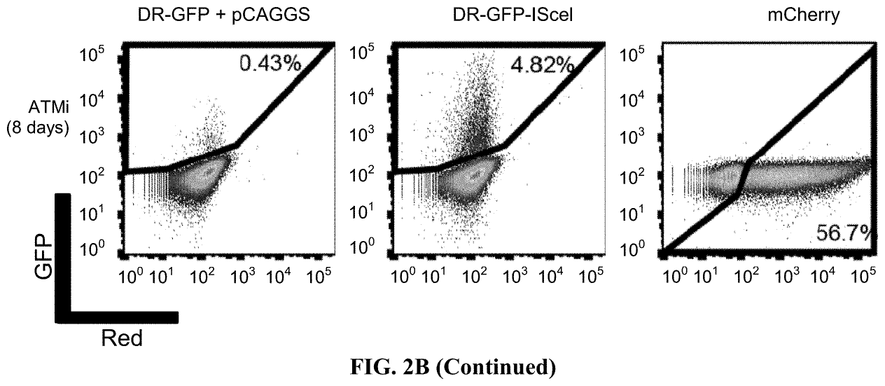

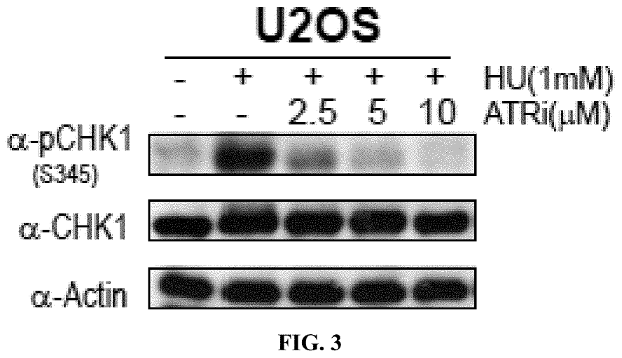

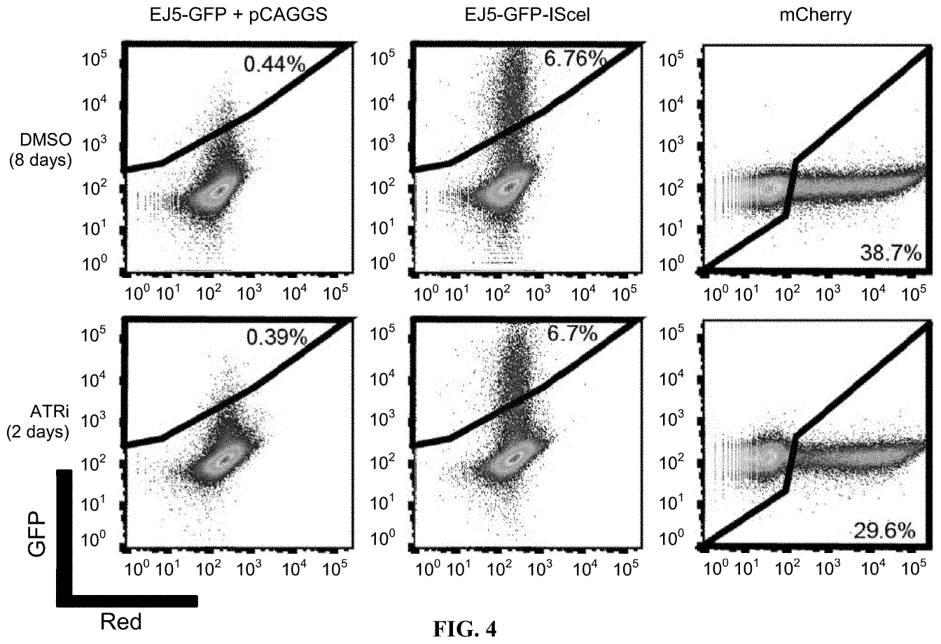

Despite extensive work investigating the roles of ATR in genome maintenance and establishing inhibitors as potential anti-cancer agents, the extent to which ATR inhibition impacts DNA repair is not well understood. ATR has been shown to control HR-mediated repair, in part, by promoting interactions between HR factors, such as BRCA1 interactions with PALB2 and TOPBP1 (Liu et al., “TOPBP1(Dpb11) Plays a Conserved Role in Homologous Recombination DNA Repair Through the Coordinated Recruitment of 53BP1(Rad9),” J. Cell Biol. 216:623-639 (2017) and Buisson et al., “Coupling of Homologous Recombination and the Checkpoint by ATR,” Mol. Cell. 65:336-346 (2017), which are hereby incorporated by reference in their entirety). Here, using the DR-GFP reporter system (Pierce et al., “XRCC3 Promotes Homology-Directed Repair of DNA Damage in Mammalian Cells,” Genes Dev. 13:2633-2638 (1999), which is hereby incorporated by reference in its entirety), it was found that long-term ATR inhibition (8 days) in U2OS cells leads to a strikingly more severe impairment in HR-mediated repair as compared to short-term ATR inhibition (FIGS. 1A, 2), and hypothesized that ATR also controls HR through mechanisms other than the regulation of protein-protein interactions. The concentration of ATR inhibitor (ATRi) used in these experiments (5 μM) resulted in almost complete ATR inhibition, as monitored by CHK1 phosphorylation in HU-treated cells (FIG. 3). Of note, chronic ATM inhibition did not result in major changes in HR-mediated repair efficiency (FIG. 1A), consistent with previous reports (Kass et al., “Double-Strand Break Repair by Homologous Recombination in Primary Mouse Somatic Cells Requires BRCA1 but not the ATM Kinase,” Proc. Natl. Acad. Sci. USA 110:5564-5569 (2013) and Chen et al., “ATM Loss Leads to Synthetic Lethality in BRCA1 BRCT Mutant Mice Associated with Exacerbated Defects in Homology-Directed Repair,” Proc. Natl. Acad. Sci. USA 114:7665-7670 (2017), which are hereby incorporated by reference in their entirety). Analysis using the EJ5-GFP (NHEJ) assay (Bennardo et al., “Alternative-NHEJ is a Mechanistically Distinct Pathway of Mammalian Chromosome Break Repair,” PLoS Genet. 4:e1000110 (2008), which is hereby incorporated by reference in its entirety) revealed that chronic ATR inhibition does not result in a significant change in NHEJ efficiency (FIGS. 1B and 4), supporting that after 8 days treatment with 5 μM of ATRi, cells are still able to utilize NHEJ-mediated repair. Furthermore, the severe reduction in HR-mediated repair upon chronic ATR inhibition was not due to major changes in the cell cycle, as it results in a minor increase in the number of S-phase cells upon chronic ATR inhibition (FIGS. 1C, 1D). Of note, DNA damage was not detectable in cells undergoing chronic ATR inhibition alone. Taken together, these findings are consistent with a pro-HR-mediated repair function for ATR. Since ATR-mediated protein-protein interactions are expected to be impaired by short-term treatment, these results suggest that the severe effect of long-term ATR inhibition on HR-mediated repair occurs through alternative mechanisms.

### Example 2—Chronic ATR Inhibition Severely Impairs HR-Mediated Repair

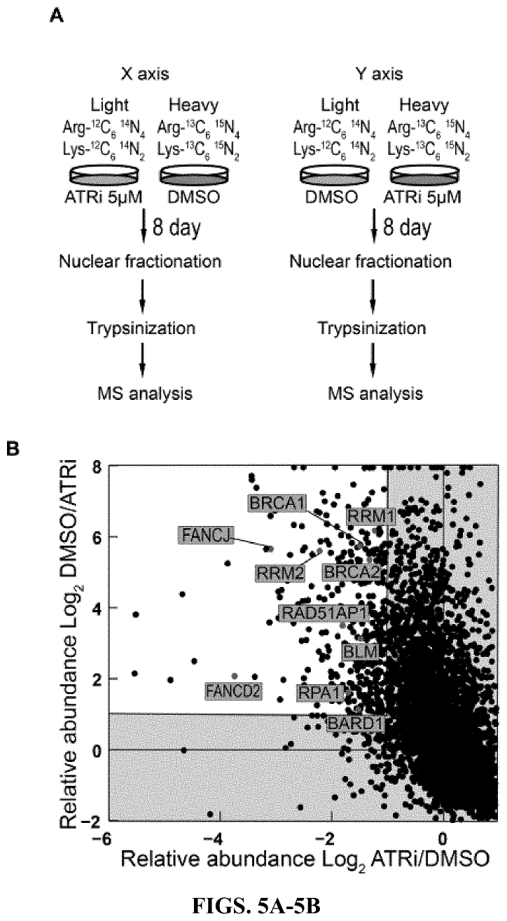

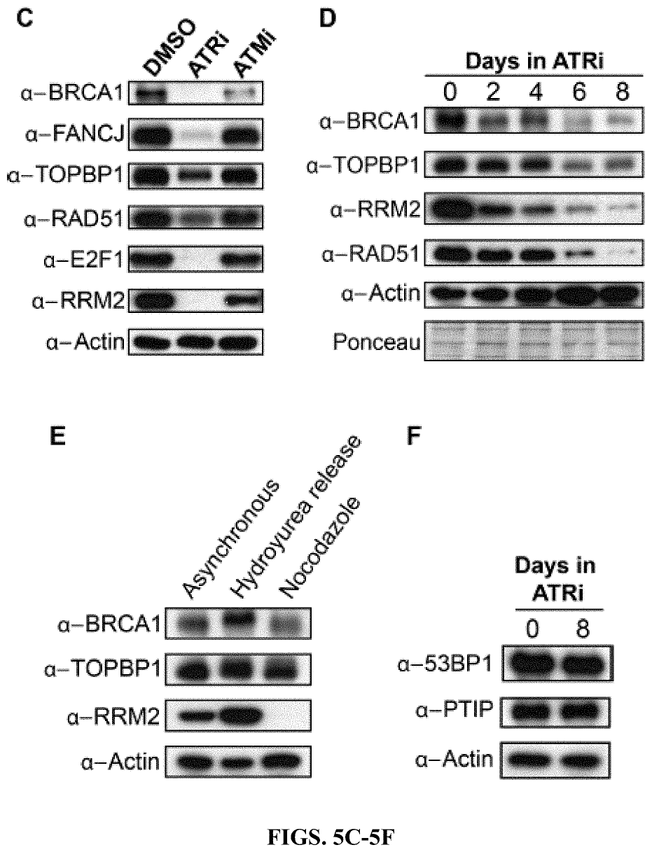

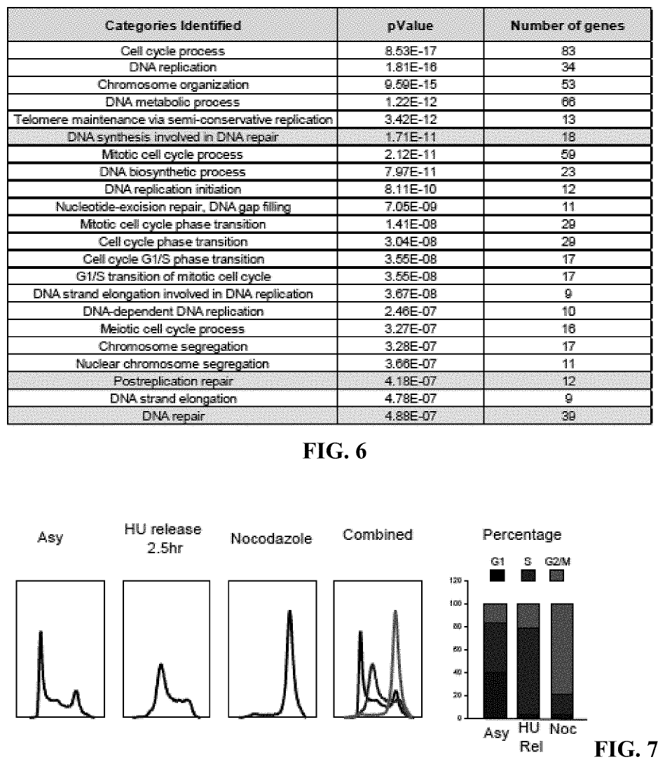

It was hypothesized that chronic ATR inhibition might strongly impair HR-mediated repair by altering the abundance of proteins required for HR. While the ATR-CHK1 pathway has been shown to control gene expression (Bertoli et al., “Control of Cell Cycle Transcription During G1 and S Phases,” Nat. Rev. Mol. Cell Biol. 14:518-528 (2013); Bertoli et al., “Sustained E2F-Dependent Transcription Is a Key Mechanism to Prevent Replication-Stress-Induced DNA Damage,” Cell Rep. 15:1412-1422 (2016); Bertoli et al., “Chk1 Inhibits E2F6 Repressor Function in Response to Replication Stress to Maintain Cell-Cycle Transcription,” Curr. Biol. 23:1629-1637 (2013); Bastos de Oliveira, “Linking DNA Replication Checkpoint to MBF Cell-Cycle Transcription Reveals a Distinct Class of G1/S Genes,” EMBO J. 31:1798-1810 (2012); and Travesa et al., “DNA Replication Stress Differentially Regulates G1/S Genes via Rad53-Dependent Inactivation of Nrm1,” EMBO J. 31:1811-1822 (2012), which are hereby incorporated by reference in their entirety), little is understood about the impact of ATR inhibition in shaping DNA repair mechanisms via control of protein abundance. To test the above hypothesis, proteomic analysis of nuclear proteins extracted from U2OS cells was performed, comparing the nuclear proteome of untreated cells with the nuclear proteome of cells treated for 8 days with 5 μM of ATRi. Stable isotope labeling was used with amino acids in cell culture (SILAC) and two experiments were performed inverting the light and heavy isotopes (FIG. 5A). Consistent with the above hypothesis, the abundance of proteins required for HR-mediated repair, including BRCA1 and FANCJ, was reduced in cells treated chronically with ATRi (FIG. 5B; Table 2). Notably, BRCA1 and FANCJ expression is regulated by E2F (Wang et al., “Regulation of BRCA1 Expression by the Rb-E2F Pathway,” J. Biol. Chem. 275:4532-4536 (2000) and Eelen et al., “Expression of the BRCA1-Interacting Protein Brip1/BACH1/FANCJ is Driven by E2F and Correlates with Human Breast Cancer Malignancy,” Oncogene 27:4233-4241 (2008), which are hereby incorporated by reference in their entirety), which in turn can be controlled by the ATR-CHK1 pathway (Bertoli et al., “Control of Cell Cycle Transcription During G1 and S Phases,” Nat. Rev. Mol. Cell Biol. 14:518-528 (2013) and Bertoli et al., “Chk1 Inhibits E2F6 Repressor Function in Response to Replication Stress to Maintain Cell-Cycle Transcription,” Curr. Biol. 23:1629-1637 (2013), which are hereby incorporated by reference in their entirety). Gene Ontology-analysis of the proteomic dataset revealed that the majority of the proteins whose abundance is reduced upon chronic ATR inhibition belong to categories associated with DNA replication and repair (FIG. 6; Table 3).

TABLE 2
Protein Abundance

| | Experiment 1 | Experiment 1 | Experiment 2 | Experiment 2 |
|---|---|---|---|---|
| | (FIG. 5B: X-axis) | (FIG. 5B: X-axis) | (FIG. 5B: Y-axis) | (FIG. 5B: Y-axis) |
| | Number of | Ratio of protein | Number of | Ratio of protein |
| | peptides | abundance | peptides | abundance |
| Protein | identified | (DMSO/ATRi) | identified | (DMSO/ATRi) |
| ACAT2 | 52 | 2.26 | 45 | 26.55 |
| ACBD7 | 4 | 4.25 | 4 | 82.08 |
| ACTL6A | 20 | 2.27 | 31 | 2.50 |
| ADD1 | 13 | 3.02 | 25 | 27.28 |
| ADD3 | 6 | 4.31 | 17 | 9.91 |
| ADI1 | 8 | 2.44 | 4 | 6.99 |
| ADNP | 31 | 2.79 | 63 | 6.06 |
| AGFG2 | 7 | 2.25 | 3 | 3.15 |
| AHDC1 | 3 | 2.52 | 4 | 9.25 |
| AIDA | 6 | 2.90 | 6 | 29.52 |
| AIM1 | 3 | 2.92 | 5 | 51.89 |
| AKT3 | 4 | 3.17 | 6 | 76.36 |
| ALDH7A1 | 90 | 2.27 | 105 | 2.50 |
| AMPH | 15 | 8.55 | 15 | 96.09 |
| ANKRD44 | 8 | 3.82 | 4 | 14.72 |
| ANKZF1 | 6 | 2.62 | 3 | 121.99 |
| ANP32E | 21 | 2.43 | 4 | 151.93 |
| ANXA3 | 28 | 2.57 | 15 | 3.63 |
| ANXA8 | 2 | 2.55 | 15 | 56.85 |
| ARHGAP12 | 12 | 4.39 | 3 | 18.78 |
| ARHGEF10 | 5 | 2.94 | 7 | 6.70 |
| ARHGEF17 | 11 | 5.75 | 7 | 18.20 |
| ARIDB1 | 21 | 2.36 | 18 | 4.47 |
| ARNT | 6 | 2.40 | 10 | 20.08 |
| ARRB2 | 3 | 3.41 | 11 | 7.99 |
| ASAP3 | 5 | 3.15 | 3 | 102.53 |
| ATAD2 | 16 | 4.00 | 29 | 2.59 |
| ATAD5 | 3 | 5.73 | 4 | 49.04 |
| ATXN3 | 2 | 3.76 | 4 | 77.62 |
| ATXN7L3B | 7 | 2.67 | 4 | 244.73 |
| BAIAP2L1 | 15 | 3.13 | 8 | 44.90 |
| BARD1 | 3 | 2.82 | 2 | 2.20 |
| BAZ2B | 4 | 2.80 | 12 | 54.27 |
| BCAM | 12 | 5.66 | 11 | 4.03 |
| BEND3 | 7 | 2.67 | 7 | 2.60 |
| BLM | 4 | 2.75 | 4 | 8.83 |
| BRCA1 | 3 | 2.82 | 4 | 53.44 |
| BRCA2 | 5 | 2.50 | 5 | 40.78 |
| BRD7 | 5 | 2.86 | 11 | 3.24 |
| BUB1B | 13 | 2.83 | 7 | 40.36 |
| C10ORF119 | 34 | 2.76 | 40 | 14.61 |
| C12ORF57 | 4 | 2.31 | 3 | 3.35 |
| C15ORF23 | 8 | 2.90 | 10 | 51.98 |
| C16ORF87 | 3 | 2.85 | 2 | 3.00 |
| C19ORF21 | 27 | 4.00 | 51 | 20.57 |
| C1ORF198 | 8 | 10.85 | 6 | 209.70 |
| C3ORF37 | 8 | 2.25 | 8 | 9.22 |
| C4ORF27 | 18 | 2.34 | 9 | 4.59 |
| CALD1 | 96 | 2.16 | 123 | 2.33 |
| CAMK1D | 2 | 5.69 | 2 | 636.31 |
| CAMK4 | 5 | 6.40 | 2 | 636.31 |
| CAP2 | 7 | 2.01 | 4 | 95.52 |
| CAPG | 44 | 2.21 | 45 | 2.04 |
| CARHSP1 | 15 | 4.67 | 10 | 19.50 |
| CASKIN2 | 3 | 45.74 | 4 | 14.02 |
| CASP7 | 16 | 2.49 | 4 | 17.26 |
| CBR3 | 7 | 3.18 | 2 | 10.91 |
| CBS | 21 | 2.11 | 12 | 26.01 |
| CBX2 | 7 | 9.06 | 4 | 50.16 |
| CEP192 | 5 | 3.12 | 5 | 84.13 |
| CEP350 | 5 | 4.52 | 3 | 5.82 |
| CFDP1 | 6 | 7.61 | 3 | 2.67 |
| CHAF1B | 5 | 2.81 | 3 | 3.70 |
| CHTF18 | 17 | 2.15 | 17 | 6.36 |
| CIT | 17 | 2.88 | 21 | 20.15 |
| CKB | 36 | 2.38 | 15 | 9.34 |
| CNN2 | 30 | 2.39 | 23 | 5.51 |
| CNN3 | 42 | 2.05 | 38 | 18.38 |
| COG6 | 3 | 2.23 | 4 | 8.94 |
| CRTC3 | 4 | 3.85 | 2 | 636.31 |
| CSE1L | 89 | 2.05 | 65 | 12.18 |
| CSNK1E | 3 | 3.70 | 2 | 41.65 |
| CSRP2 | 22 | 2.31 | 24 | 5.45 |
| CTDSPL2 | 4 | 2.30 | 15 | 2.45 |
| CUL4B | 23 | 2.25 | 33 | 3.03 |
| CYTH1 | 4 | 2.02 | 4 | 2.74 |
| DCUN1D1 | 10 | 2.62 | 6 | 10.16 |
| DDAH2 | 14 | 4.46 | 12 | 6.27 |
| DENND4C | 8 | 2.28 | 8 | 2.96 |
| DIS3L | 3 | 2.84 | 4 | 5.05 |
| DLGAP5 | 15 | 6.41 | 16 | 80.46 |
| DNAJC21 | 10 | 2.96 | 5 | 3.36 |
| DNAJC9 | 34 | 3.17 | 26 | 4.64 |
| DNM1 | 25 | 5.83 | 27 | 8.41 |
| DNMT1 | 59 | 2.95 | 72 | 5.21 |
| DPYSL2 | 101 | 3.16 | 86 | 38.93 |
| DPYSL3 | 67 | 2.12 | 70 | 28.05 |
| DPYSL4 | 11 | 2.18 | 7 | 39.96 |
| DPYSL5 | 4 | 3.72 | 8 | 141.71 |
| DSN1 | 2 | 2.10 | 12 | 8.54 |
| DUT | 34 | 3.72 | 35 | 9.15 |
| EDIL3 | 5 | 2.61 | 61 | 3.95 |
| EEF2K | 5 | 3.50 | 3 | 13.02 |
| ELAVL2 | 6 | 2.28 | 10 | 2.78 |
| ELP4 | 3 | 2.35 | 2 | 7.65 |
| ERBB2IP | 30 | 2.12 | 18 | 11.99 |
| ERCC6L | 26 | 3.64 | 28 | 2.57 |
| ESPL1 | 7 | 2.24 | 3 | 2.19 |
| FANCD2 | 4 | 13.38 | 14 | 4.22 |
| FANCJ | 5 | 8.54 | 6 | 50.24 |
| FDPS | 28 | 2.39 | 6 | 14.37 |
| FHL1 | 53 | 2.75 | 33 | 33.28 |
| FKBPL | 4 | 2.62 | 5 | 61.40 |
| FNTA | 7 | 2.65 | 3 | 25.82 |
| FPGS | 2 | 5.39 | 6 | 4.76 |
| FTO | 14 | 2.40 | 9 | 49.23 |
| GABPA | 14 | 2.10 | 12 | 2.15 |
| GGA2 | 9 | 2.04 | 5 | 46.55 |
| GINS1 | 3 | 22.10 | 5 | 5.63 |
| GINS3 | 3 | 4.14 | 6 | 54.99 |
| GINS4 | 5 | 6.44 | 13 | 11.84 |
| GLI2 | 5 | 4.48 | 6 | 150.29 |
| GSDMD | 21 | 4.22 | 36 | 6.13 |
| GSTCD | 3 | 3.73 | 3 | 23.15 |
| H1FX | 40 | 3.18 | 38 | 2.07 |
| HAT1 | 19 | 2.22 | 18 | 67.24 |
| HAUS6 | 6 | 2.81 | 8 | 5.33 |
| HAUS8 | 5 | 4.70 | 9 | 6.80 |
| HDAC7 | 2 | 2.08 | 3 | 636.31 |
| HEATR6 | 16 | 2.76 | 5 | 17.44 |
| HEG1 | 3 | 3.07 | 9 | 3.36 |
| HIP1 | 17 | 2.09 | 31 | 3.72 |
| HIRIP3 | 3 | 4.74 | 12 | 14.95 |
| HIST1H1A | 20 | 7.47 | 37 | 22.61 |
| HIST1H1C | 19 | 3.90 | 32 | 3.45 |
| HMBS | 6 | 3.04 | 2 | 2.58 |
| HMGN5 | 12 | 2.11 | 13 | 17.88 |
| HN1L | 46 | 2.34 | 30 | 14.35 |
| HSP90AB6P | 2 | 2.56 | 2 | 2.79 |
| HSPB1 | 201 | 3.42 | 135 | 15.70 |
| HSPB11 | 2 | 2.16 | 6 | 37.71 |
| IAH1 | 11 | 4.20 | 3 | 23.82 |
| IL18 | 29 | 2.88 | 14 | 2.20 |
| INPP5F | 3 | 2.10 | 2 | 15.33 |
| IPO9 | 22 | 2.24 | 4 | 27.64 |
| IQGAP3 | 7 | 25.67 | 8 | 20.85 |
| IRS1 | 3 | 10.80 | 8 | 194.29 |
| ISOCI | 6 | 2.07 | 2 | 43.22 |
| ITPK1 | 4 | 2.45 | 2 | 6.74 |
| ITPKA | 2 | 8.68 | 3 | 12.01 |
| ITSN1 | 10 | 2.07 | 11 | 55.70 |
| JUP | 6 | 2.51 | 21 | 20.02 |
| KANK2 | 4 | 2.54 | 4 | 132.01 |
| KDM1A | 14 | 2.15 | 26 | 3.61 |
| KIAA0101 | 2 | 7.31 | 8 | 3.91 |
| KIAA1279 | 5 | 2.36 | 16 | 7.56 |
| KIAA1522 | 14 | 2.19 | 18 | 14.01 |
| KIAA1524 | 21 | 4.14 | 7 | 6.95 |
| KIAA1598 | 35 | 2.48 | 21 | 3.68 |
| KIF11 | 54 | 3.37 | 25 | 37.82 |
| KIF15 | 18 | 3.44 | 9 | 46.96 |
| KIF20B | 4 | 2.89 | 18 | 9.05 |
| KIF4A | 65 | 2.96 | 66 | 5.74 |
| KIF5C | 5 | 3.24 | 2 | 64.14 |
| KNTC1 | 7 | 3.70 | 4 | 22.31 |
| L1CAM | 2 | 2.06 | 23 | 3.05 |
| L3MBTL3 | 4 | 2.48 | 3 | 3.05 |
| LANCL1 | 4 | 2.03 | 3 | 4.32 |
| LCOR | 7 | 2.59 | 7 | 67.52 |
| LIG1 | 30 | 3.58 | 30 | 16.51 |
| LIMCH1 | 11 | 2.25 | 2 | 10.46 |
| LLGL1 | 11 | 2.37 | 13 | 45.74 |
| LMCD1 | 5 | 2.51 | 8 | 255.93 |
| LMO7 | 40 | 2.49 | 29 | 16.29 |
| LPP | 25 | 2.19 | 30 | 8.27 |
| LRBA | 2 | 29.70 | 5 | 3.90 |
| LSM14B | 8 | 2.66 | 7 | 9.38 |
| LUC7L2 | 52 | 2.18 | 33 | 2.48 |
| MAGI3 | 2 | 3.77 | 5 | 31.05 |
| MAP3K2 | 12 | 2.22 | 6 | 44.25 |
| MASTL | 7 | 4.26 | 4 | 59.51 |
| MAT1A | 4 | 46.41 | 2 | 4.43 |
| MCM10 | 2 | 2.32 | 3 | 2.01 |
| MCM2 | 56 | 2.71 | 70 | 5.07 |
| MCM3 | 129 | 2.48 | 126 | 2.96 |
| MCM4 | 137 | 2.91 | 160 | 4.72 |
| MCM5 | 42 | 2.47 | 51 | 3.37 |
| MCM6 | 84 | 2.17 | 98 | 4.60 |
| MCM7 | 72 | 2.68 | 58 | 2.76 |
| METT10D | 11 | 3.07 | 6 | 12.11 |
| MGMT | 2 | 2.13 | 3 | 95.99 |
| MKL1 | 5 | 2.17 | 4 | 47.49 |
| MKL2 | 18 | 4.28 | 18 | 97.19 |
| MLF1IP | 2 | 3.20 | 6 | 3.69 |
| MLH1 | 20 | 2.08 | 41 | 7.02 |
| MMS19 | 15 | 2.47 | 10 | 2.13 |
| MOCOS | 5 | 2.16 | 4 | 3.16 |
| MOV10 | 24 | 2.79 | 44 | 8.73 |
| MRC2 | 38 | 2.95 | 59 | 2.24 |
| MRI1 | 12 | 5.10 | 6 | 45.38 |
| MSH2 | 32 | 2.31 | 51 | 3.68 |
| MSH6 | 51 | 2.32 | 61 | 3.93 |
| MTA3 | 4 | 5.37 | 7 | 18.47 |
| MTUS1 | 4 | 4.66 | 9 | 102.33 |
| MYCBP2 | 8 | 2.91 | 5 | 8.97 |
| MYO18B | 12 | 2.14 | 5 | 83.61 |
| N4BP1 | 2 | 3.17 | 4 | 47.78 |
| NAP1L1 | 82 | 2.17 | 42 | 12.29 |
| NASP | 160 | 2.86 | 89 | 37.90 |
| NCAPD2 | 65 | 2.90 | 100 | 6.71 |
| NCAPD3 | 6 | 2.77 | 16 | 25.58 |
| NCAPG | 20 | 2.76 | 14 | 5.79 |
| NCAPH | 44 | 2.65 | 44 | 8.89 |
| NCAPH2 | 9 | 2.53 | 13 | 45.91 |
| NCOA1 | 4 | 2.07 | 5 | 38.82 |
| NCOA2 | 3 | 2.18 | 4 | 69.94 |
| NCOR2 | 17 | 2.48 | 38 | 47.43 |
| NDC80 | 20 | 5.50 | 34 | 8.54 |
| NDE1 | 20 | 2.33 | 6 | 9.64 |
| NDNL2 | 2 | 4.81 | 2 | 2.38 |
| NFIC | 16 | 2.59 | 18 | 2.46 |
| NFKBIL2 | 4 | 2.04 | 18 | 13.72 |
| NHEJ1 | 3 | 2.24 | 4 | 26.11 |
| NNMT | 49 | 2.31 | 38 | 2.99 |
| NRGN | 5 | 2.98 | 3 | 5.12 |
| NTM | 7 | 5.80 | 2 | 78.42 |
| NUCKS1 | 9 | 3.26 | 8 | 3.06 |
| NUDCD2 | 27 | 2.00 | 12 | 8.46 |
| NUDCD3 | 12 | 2.36 | 2 | 6.33 |
| NUDT1 | 14 | 3.33 | 13 | 3.23 |
| NUDT15 | 5 | 3.18 | 6 | 6.90 |
| NUF2 | 14 | 4.05 | 10 | 4.59 |
| NUSAP1 | 13 | 2.23 | 6 | 9.53 |
| NXN | 8 | 2.56 | 2 | 4.55 |
| ODZ2 | 2 | 2.75 | 9 | 70.55 |
| ODZ3 | 2 | 3.26 | 14 | 114.41 |
| OSBPL11 | 8 | 2.20 | 7 | 7.04 |
| PALLD | 74 | 5.60 | 50 | 51.80 |
| PALM | 3 | 2.27 | 8 | 7.10 |
| PARG | 7 | 3.57 | 5 | 65.94 |
| PBK | 15 | 5.57 | 14 | 18.35 |
| PCBP4 | 6 | 5.14 | 3 | 24.39 |
| PDE5A | 11 | 4.21 | 2 | 7.53 |
| PDLIM1 | 4 | 2.39 | 2 | 636.31 |
| PDXK | 31 | 2.07 | 18 | 8.34 |
| PHC3 | 5 | 2.18 | 8 | 10.53 |
| PHGDH | 107 | 2.59 | 106 | 8.57 |
| PHLDB2 | 15 | 2.41 | 13 | 14.06 |
| PIH1D1 | 3 | 2.95 | 3 | 27.65 |
| PKIG | 3 | 7.77 | 3 | 13.10 |
| PLCB3 | 21 | 2.45 | 14 | 2.12 |
| PLCD1 | 4 | 2.28 | 5 | 33.85 |
| PLEKHA5 | 36 | 2.42 | 23 | 5.17 |
| PLS1 | 34 | 2.22 | 16 | 3.08 |
| PMS2 | 26 | 2.53 | 7 | 14.92 |
| POLA1 | 14 | 2.30 | 16 | 40.32 |
| POLA2 | 24 | 4.13 | 10 | 34.13 |
| POLD1 | 32 | 3.90 | 27 | 120.20 |
| POLD2 | 21 | 3.53 | 23 | 43.12 |
| POLD3 | 10 | 4.05 | 5 | 46.64 |
| POLE | 12 | 3.80 | 21 | 4.36 |
| PPM1B | 10 | 3.23 | 8 | 6.42 |
| PPME1 | 42 | 2.12 | 19 | 15.89 |
| PPP1R2 | 8 | 10.42 | 5 | 4.15 |
| PPP2R5D | 8 | 4.08 | 7 | 14.62 |
| PPP4R1 | 33 | 2.16 | 22 | 31.01 |
| PPP4R2 | 10 | 3.30 | 9 | 14.68 |
| PRIM2 | 10 | 3.06 | 8 | 176.94 |
| PRKAR2B | 6 | 2.29 | 6 | 12.98 |
| PRPSAP1 | 12 | 2.19 | 11 | 2.58 |
| PRR12 | 12 | 2.67 | 14 | 2.91 |
| PSMA2 | 38 | 2.39 | 35 | 2.46 |
| PSPH | 12 | 2.62 | 5 | 15.60 |
| PTK7 | 18 | 3.29 | 53 | 7.69 |
| PTMA | 53 | 2.59 | 11 | 9.43 |
| PYGM | 2 | 2.42 | 6 | 28.45 |
| RAD51AP1 | 4 | 3.50 | 2 | 11.28 |
| RB1 | 15 | 3.14 | 14 | 3.65 |
| RBP1 | 16 | 3.11 | 21 | 56.66 |
| RCC2 | 48 | 2.12 | 76 | 6.41 |
| RECQL4 | 12 | 4.60 | 24 | 58.44 |
| RFC1 | 44 | 2.21 | 27 | 2.38 |
| RFC2 | 19 | 2.44 | 23 | 2.59 |
| RFC3 | 41 | 2.16 | 49 | 2.37 |
| RFC4 | 35 | 2.81 | 48 | 2.84 |
| RFC5 | 18 | 3.42 | 21 | 3.26 |
| RNASEH2B | 4 | 2.74 | 9 | 11.78 |
| RNF123 | 3 | 3.71 | 2 | 3.36 |
| RPA1 | 26 | 3.30 | 51 | 2.65 |
| RPS6KB1 | 7 | 2.30 | 7 | 50.18 |
| RPS6KB2 | 4 | 3.25 | 2 | 35.04 |
| RRM1 | 21 | 2.35 | 26 | 72.06 |
| RRM2 | 6 | 4.66 | 5 | 48.54 |
| RSBN1L | 14 | 2.62 | 20 | 8.67 |
| S100A2 | 8 | 3.54 | 11 | 5.24 |
| SAC3D1 | 5 | 2.30 | 10 | 31.88 |
| SAMD1 | 8 | 2.14 | 5 | 52.78 |
| SCRN2 | 8 | 2.10 | 12 | 9.70 |
| SETDB1 | 4 | 2.03 | 6 | 2.17 |
| SH3BGRL | 46 | 2.20 | 13 | 4.48 |
| SHKBP1 | 9 | 2.55 | 9 | 2.15 |
| SHMT1 | 8 | 6.95 | 12 | 27.15 |
| SHROOM3 | 6 | 4.82 | 7 | 105.25 |
| SKA3 | 11 | 4.11 | 5 | 35.49 |
| SMARCC1 | 37 | 3.24 | 43 | 2.52 |
| SMC2 | 100 | 2.79 | 78 | 2.35 |
| SMC4 | 81 | 2.86 | 103 | 4.16 |
| SMYD2 | 4 | 14.60 | 7 | 37.92 |
| SNCG | 9 | 2.90 | 2 | 41.06 |
| SORBS3 | 8 | 3.50 | 6 | 3.54 |
| SP3 | 6 | 2.52 | 6 | 2.28 |
| SPAG5 | 30 | 2.52 | 33 | 26.76 |
| SPC24 | 6 | 7.78 | 6 | 21.96 |
| SPC25 | 13 | 7.65 | 24 | 19.60 |
| SRGAP2 | 11 | 2.23 | 12 | 8.77 |
| SSU72 | 6 | 2.22 | 5 | 9.75 |
| STAG1 | 3 | 2.69 | 24 | 3.55 |
| STMN2 | 37 | 3.45 | 13 | 14.38 |
| STRN4 | 7 | 2.05 | 10 | 12.36 |
| STXBP4 | 8 | 2.15 | 3 | 8.92 |
| SUGT1 | 24 | 2.15 | 19 | 11.78 |
| SUN2 | 9 | 2.73 | 40 | 5.98 |
| SYNE2 | 9 | 6.08 | 55 | 17.09 |
| SYNJ2 | 11 | 2.27 | 6 | 26.67 |
| TACC3 | 16 | 2.08 | 10 | 32.15 |
| TAF4 | 18 | 2.17 | 10 | 10.03 |
| TAGLN | 25 | 2.78 | 10 | 4.19 |
| TAGLN2 | 184 | 2.41 | 33 | 11.90 |
| TBC1D1 | 13 | 2.46 | 9 | 9.15 |
| TBCB | 40 | 2.05 | 17 | 5.36 |
| TBCD | 30 | 2.26 | 26 | 4.73 |
| TBCE | 17 | 2.57 | 11 | 2.67 |
| TBL1X | 2 | 2.39 | 2 | 9.62 |
| TBL1XR1 | 18 | 2.31 | 14 | 10.96 |
| TBL1Y | 5 | 2.68 | 10 | 104.42 |
| TCEB1 | 29 | 3.18 | 26 | 2.96 |
| TCP11L1 | 3 | 2.24 | 7 | 34.11 |
| TGFB1I1 | 3 | 2.22 | 5 | 12.31 |
| TGFB2 | 2 | 8.11 | 5 | 106.19 |
| TK1 | 10 | 3.06 | 14 | 9.94 |
| TLK1 | 6 | 3.74 | 8 | 4.19 |
| TMPO | 98 | 2.60 | 110 | 5.33 |
| TMSB10 | 43 | 2.16 | 19 | 2.38 |
| TMSL3 | 25 | 5.32 | 21 | 10.50 |
| TNRC18 | 2 | 5.64 | 2 | 3.51 |
| TNS1 | 20 | 2.68 | 16 | 120.53 |
| TNS3 | 19 | 10.20 | 19 | 166.60 |
| TOR3A | 3 | 3.25 | 6 | 2.75 |
| TPM1 | 24 | 4.01 | 19 | 3.61 |
| TPM2 | 68 | 2.98 | 49 | 3.38 |
| TRIM65 | 2 | 2.76 | 5 | 27.23 |
| TSEN15 | 5 | 2.40 | 4 | 16.44 |
| TTC37 | 15 | 2.53 | 12 | 5.36 |
| TUBB | 86 | 2.21 | 40 | 7.65 |
| TUBB2B | 158 | 2.20 | 127 | 11.94 |
| TYMS | 25 | 6.72 | 56 | 44.84 |
| UBA2 | 43 | 2.22 | 22 | 74.90 |
| UBE2K | 27 | 2.12 | 16 | 14.39 |
| UBE2O | 12 | 2.28 | 13 | 6.80 |
| UBE3C | 3 | 2.09 | 5 | 161.43 |
| UBR7 | 17 | 2.43 | 3 | 636.31 |
| UCKL1 | 4 | 3.27 | 3 | 2.18 |
| UNC119B | 12 | 2.14 | 8 | 38.51 |
| UNK | 6 | 3.64 | 5 | 38.22 |
| USP11 | 19 | 2.64 | 12 | 50.33 |
| USP13 | 8 | 4.52 | 12 | 9.78 |
| USP48 | 20 | 2.24 | 25 | 2.35 |
| UTRN | 54 | 2.40 | 73 | 4.38 |
| WDHD1 | 38 | 3.52 | 39 | 25.21 |
| WHSC1 | 14 | 4.03 | 31 | 4.15 |
| XRN1 | 13 | 3.19 | 3 | 2.01 |
| YWHAH | 22 | 2.20 | 18 | 5.51 |
| ZBTB2 | 5 | 4.02 | 6 | 2.78 |
| ZC3H7A | 17 | 2.27 | 19 | 4.11 |
| ZMYM3 | 31 | 3.26 | 48 | 6.04 |
| ZNF507 | 4 | 2.02 | 2 | 4.02 |
| ZNF608 | 5 | G. Pacchiana, | 15 | 127.87 |

TABLE 3
GO Analysis

| Categories | |
|---|---|
| Identified | Proteins |
| Cell Cycle Process | DSN1, RFC4, RFC5, RFC2, RFC3, KIF20B, RFC1, BRCA2, |
| | NUF2, BRCA1, PRIM2, LIG1, SPAG5, NCAPH, MLH1, SUN2, |
| | SMC2, FANCD2, NASP, TACC3, CIT, ESPL1, RPA1, MSH6, |
| | SPC24, NCAPG, FANCJ, IQGAP3, SPC25, NCAPD3, BLM, |
| | PCBP4, MASTL, KIF11, MCM4, MCM3, MCM6, MCM5, |
| | MCM2, KIF4A, MLF1IP, KNTC1, SMC4, LLGL1, MSH2, |
| | NDE1, HAUS8, ERCC6L, MCM7, BARD1, DLGAP5, MCMBP, |
| | TUBB, NCAPD2, PHGDH, ARHGEF10, HAUS6, MCM10, |
| | KIAA0101, KNSTRN, PPME1, TYMS, RPS6KB1, POLA2, |
| | BUB IB, TGFB2, RB1, TBCE, GINS1, CEP 192, NDC80, |
| | NUSAP1, CUL4B, CSNK1E, POLE, POLD2, POLDI, POLA1, |
| | POLD3, PRKAR2B, RCC2, STAG1, RRM2 |
| DNA Replication | RFC4, RFC5, BARD1, MCM7, RFC2, RFC3, MCMBP, RFC1, |
| | BRCA1, CHAF1B, MCM10, NFIC, KIAA0101, WDHD1, |
| | NAP1L1, NASP, RPA1, POLA2, FANCJ, DUT, RECQL4, |
| | CHTF18, BLM, MCM4, MCM3, MCM6, POLE, MCM5, POLD2, |
| | POLD1, MCM2, POLA1, RRM1, RRM2 |
| Chromosome | DSN1, RFC4, RFC5, HAT1, RFC2, RFC3, RFC1, BRCA2, |
| Organization | NUF2, PRIM2, SPAG5, NCAPH, MLH1, FANCD2, SMC2, |
| | ESPL1, RPA1, MSH6, SPC24, NCAPH2, NCAPG, FANCJ, |
| | RECQL4, SPC25, NCAPD3, BLM, MCM4, MCM6, MCM2, |
| | MLF1IP, KNTC1, SMC4, NDE1, MSH2, ERCC6L, MCM7, |
| | MCMBP, NCAPD2, KNSTRN, POLA2, BUB1B, GINS1, |
| | NUSAP1, NDC80, GINS4, CUL4B, POLE, POLD2, POLD1, |
| | POLA1, POLD3, STAG1, RCC2 |
| DNA Metabolic | RFC4, RFC5, RFC2, RFC3, PMS2, RFC1, BRCA2, TK1, |
| Process | BRCA1, GINS3, PRIM2, LIG1, CHAF1B, NDNL2, MLH1, |
| | FANCD2, MMS19, NAP1L1, ATXN3, NASP, NUDT1, NHEJ1, |
| | RPA1, MSH6, FANCJ, FTO, DUT, RECQL4, WHSC1, BLM, |
| | TONSL, MCM4, MCM3, MCM6, MCM5, NUCKS1, MCM2, |
| | RAD51AP1, ACTL6A, MSH2, BARD1, MCM7, MCMBP, |
| | MCM10, NFIC, KIAA0101, WDHD1, TYMS, POLA2, NUDT15, |
| | DNMT1, CHTF18, BEND3, GINS1, GINS4, CUL4B, CSNK1E, |
| | POLE, POLD2, POLD1, POLA1, POLD3, CBS, MGMT, RRM1, |
| | RRM2 |
| Telomere | POLA2, RFC4, RFC5, RFC2, RFC3, RFC1, PRIM2, POLE, |
| Maintenance via | POLD2, POLD1, POLA1, POLD3, RPA1 |
| Semi-Conservative | |
| Replication | |
| DNA Synthesis | FANCJ, RFC4, RFC5, BARD1, RFC2, RFC3, RFC1, BRCA2, |
| Proteins Involved in | BRCA1, BLM, POLE, POLD2, KIAA0101, POLD1, POLA1, |
| DNA Repair | POLD3, RAD51AP1, RPA1 |
| Mitotic Cell Cycle | DSN1, BRCA2, NUF2, BRCA1, PRIM2, LIG1, SPAG5, NCAPH, |
| Process | SUN2, SMC2, NASP, CIT, TACC3, ESPL1, RPA1, NCAPG, |
| | IQGAP3, SPC25, NCAPD3, BLM, PCBP4, MASTL, MCM4, |
| | KIF11, MCM3, MCM6, MCM5, MCM2, KIF4A, KNTC1, SMC4, |
| | HAUS8, NDE1, MSH2, MCM7, DLGAP5, TUBB, NCAPD2, |
| | ARHGEF10, MCM10, HAUS6, KNSTRN, PPME1, TYMS, |
| | RPS6KB1, POLA2, BUB1B, RB1, TBCE, GINS1, CEP192, |
| | NDC80, NUSAP1, CUL4B, CSNK1E , POLE , POLA1, |
| | PRKAR2B, RRM2 |
| DNA Biosynthetic | POLA2, FANCJ, RFC4, BARD1, RFC5, RFC2, RFC3, RFC1, |
| Process | TK1, BRCA2, BRCA1, BLM, PRIM2, LIG1, POLE, POLD2, |
| | KIAA0101, POLD1, POLA1, POLD3, RAD51AP1, TYMS, |
| | RPA1 |
| DNA Replication | MCM4, POLA2, MCM3, MCM6, MCM10, POLE, MCM5, |
| Initiation | MCM7, MCM2, POLA1, PRIM2, and GINS4. |
| Nucleotide-Excision | RFC4, POLE, RFC5, POLD2, RFC2, POLD1, RFC3, RFC1, |
| Repair and DNA | POLD3, RPA1, LIG1 |
| Gap Filling | |
| Mitotic Cell Cycle | NDE1, HAUS8, MCM7, TUBB, PRIM2, MCM10, HAUS6, |
| Phase Transition | NASP, TACC3, CIT, PPME1, RPA1, RPS6KB1, POLA2, |
| | IQGAP3, BUB1B, RB1, CEP192, MASTL, CUL4B, CSNK1E, |
| | MCM4, MCM3, MCM6, POLE, MCM5, MCM2, POLA1, |
| | PRKAR2B |
| Cell Cycle Phase | HAUS8, NDE1, MCM7, TUBB, PRIM2, HAUS6, MCM10, |
| Transition | NASP, TACC3, CIT, PPME1, RPA1, RPS6KB1, POLA2, |
| | IQGAP3, BUB1B, RB1, CEP192, MASTL, CSNK1E, CUL4B, |
| | MCM4, MCM3, MCM6, POLE, MCM5, MCM2, POLA1, |
| | PRKAR2B |
| Cell Cycle G1/S | POLA2, IQGAP3, MCM7, RB1, PRIM2, CUL4B, MCM4, |
| Phase Transition | MCM3, MCM6, MCM10, POLE, MCM5, MCM2, POLA1, |
| | NASP, RPA1, RPS6KB1 |
| G1/S Transition of | POLA2, IQGAP3, MCM7, RB1, PRIM2, CUL4B, MCM4, |
| Mitotic Cell Cycle | MCM3, MCM6, MCM10, POLE, MCM5, MCM2, POLA1, |
| | NASP, RPA1, RPS6KB1 |
| DNA Strand | RFC4, POLE, POLD2, RFC3, POLA1, GINS3, GINS1, LIG1, |
| Elongation Involved | GINS4 |
| in DNA Replication | |
| DNA-Dependent | RFC4, RFC5, RFC2, POLD1, WDHD1, RFC3, MCMBP, POLA1, |
| DNA Replication | RFC1, RPA1 |
| Meiotic Cell Cycle | DSN1, FANCJ, MSH2, BUB1B, NCAPD3, BRCA2, NUF2, |
| Process | NCAPD2, MASTL, NCAPH, MLH1, FANCD2, SMC2, ESPL1, |
| | SMC4, MSH6 |
| Chromosome | DSN1, NDE1, SPC25, NUF2, BRCA1, NUSAP1, NDC80, SKA3, |
| Segregation | SPAG5, KIF11, NCAPH, MLH1, SMC2, MMS19, KNSTRN, |
| | ESPL1, SMC4 |
| Nuclear | NCAPH, DSN1, MLH1, SMC2, KNSTRN, NUF2, ESPL1, |
| Chromosome | SMC4, NUSAP1, NDC80, SPAG5 |
| Segregation | |
| Postreplication | MSH2, RFC4, RFC5, POLD2, KIAA0101, RFC2, POLD1, RFC3, |
| Repair | RFC1, POLD3, BRCA1, RPA1 |
| DNA Strand | RFC4, POLE, POLD2, RFC3, POLA1, GINS3, GINS1, LIG1, |
| Elongation | GINS4 |
| DNA Repair | ACTL6A, RFC4, MSH2, BARD1, RFC5, RFC2, PMS2, RFC3, |
| | RFC1, BRCA2, BRCA1, LIG1, CHAF1B, NDNL2, MLH1, |
| | FANCD2, MMS19, KIAA0101, WDHD1, ATXN3, NUDT1, |
| | NHEJ1, MSH6, RPA1, FANCJ, RECQL4, WHSCI, BLM, |
| | TONSL, GINS4, CUL4B, CSNK1E, POLE, POLD2, NUCKS1, |
| | POLD1, POLA1, POLD3, RAD51AP1 |

Down-regulated proteins include many known E2F targets, such as RRM2, RFCs, MCMs and POLE. Western blot (WB) analysis validated these findings (FIG. 5C). Moreover, protein levels of RAD51 and TOPBP1, two other E2F targets required for HR-mediated repair (Liu et al., “TOPBP1(Dpb11) Plays a Conserved Role in Homologous Recombination DNA Repair Through the Coordinated Recruitment of 53BP1(Rad9),” J. Cell Biol. 216: 623-639 (2017) and Baumann et al., “Role of the Human RAD51 Protein in Homologous Recombination and Double-Stranded-Break Repair,” Trends Biochem. Sci. 23:247-251 (1998), which are hereby incorporated by reference in their entirety), were also reduced upon chronic ATR inhibition (FIG. 5C). Time-course analysis revealed that minor changes in protein abundance could be observed after 2 and 4 days of continuous treatment with ATRi, and that the changes become more striking after 6 and 8 days of chronic drug treatment (FIG. 5D). Taken together, these findings suggest a model that ATR signaling occurring physiologically during every cell cycle is important to maintain the abundance of key proteins required for HR-mediated repair.

The observed changes in protein abundance are not due to cell cycle changes since chronic treatment with 5 μM of ATRi leads only to minor changes in cell cycle distribution, as seen by the slight increase in S phase cells (FIG. 1C). Importantly, the reduced expression of HR factors upon chronic ATRi treatment cannot be attributed to such slight increase in S phase cells since cells in S phase actually express more HR factors and E2F targets (FIGS. 5E and 7). Moreover, while the abundance of several proteins involved in HR-mediated repair is severely reduced upon chronic ATR inhibition, changes in the abundance of the NHEJ factors 53BP1 and PTIP were minimal (FIG. 5F). This finding is consistent with the result using the EJ5-GFP assay showing that chronic ATR inhibition does not impair NHEJ efficiency (FIG. 1B). Collectively, the findings above show that chronic ATR inhibition has a major impact on the abundance of key proteins required for HR-mediated repair, which is correlated with the severe reduction in HR-mediated repair efficiency observed in U2OS cells.

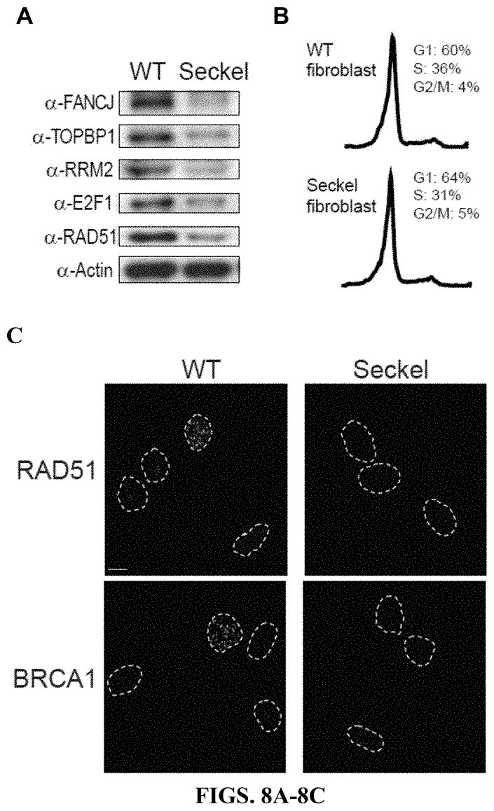

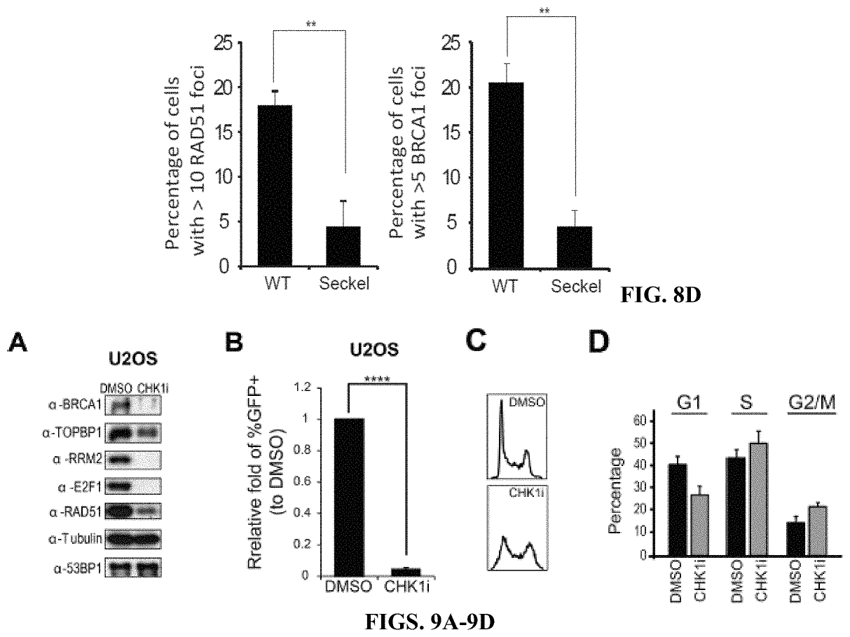

To investigate whether the role of ATR in maintaining the abundance of HR-mediated repair factors is consistently observed in a physiological setting, a Seckel syndrome patient cell line containing the A210G mutation in ATR that generates a splicing defect was investigated (O'Driscoll et al., “A Splicing Mutation Affecting Expression of Ataxia-Telangiectasia and Rad3-Related Protein (ATR) Results in Seckel Syndrome,” Nat. Genet. 33:497-501 (2003), which is hereby incorporated by reference in its entirety). Analysis of a primary fibroblast cell line GM18366, which has attenuated ATR signaling (Stokes et al., “G. Pacchiana, R. Cahoon, and T. Zangrilloabundance of HR-mediated repair factors compared to control fibroblasts (FIG. 8A). Although BRCA1 was not detected in WB for technical reasons, the abundance of FANCJ, TOPBP1, and RAD51 was decreased. This Seckel cell line did not show any major differences in cell cycle distribution, consistent with a previous report (Wilsker et al., “Loss of Ataxia Telangiectasia Mutated- and Rad3-Related Function Potentiates the Effects of Chemotherapeutic Drugs on Cancer Cell Survival,” Mol. Cancer Ther. 6:1406-1413 (2007), which is hereby incorporated by reference in its entirety), indicating that the abundance changes are not due to effects on the cell cycle (FIG. 8B). To examine the ability of Seckel cells to utilize HR-mediated repair, BRCA1 and RAD51 foci were imaged after irradiation (IR) treatment and a significant reduction in RAD51 and BRCA1 foci formation post IR was observed (FIGS. 8C and 8D). The results above reveal similarities between these Seckel cells with the U2OS cells chronically treated with ATRi. In both cases, the abundance of important HR-mediated repair factors is reduced and markers of HR-mediated repair are impaired (Casper et al., Chromosomal Instability at Common Fragile Sites in Seckel Syndrome,” Am. J. Hum. Genet. 75:654-660 (2004) and Alderton et al., “Seckel Syndrome Exhibits Cellular Features Demonstrating Defects in the ATR-Signaling Pathway,” Hum. Mol. Genet. 13:3127-3138 (2004), which are hereby incorporated by reference in their entirety). Overall, these findings support a key role for ATR in maintaining the abundance of HR-mediated repair factors and suggest that the severe impairment in HR-mediated repair upon chronic ATR inhibition is caused by depletion of key components of the HR-mediated repair machinery.

### Example 3—ATR Controls HR Factor Abundance Through CHK1-Mediated Transcription

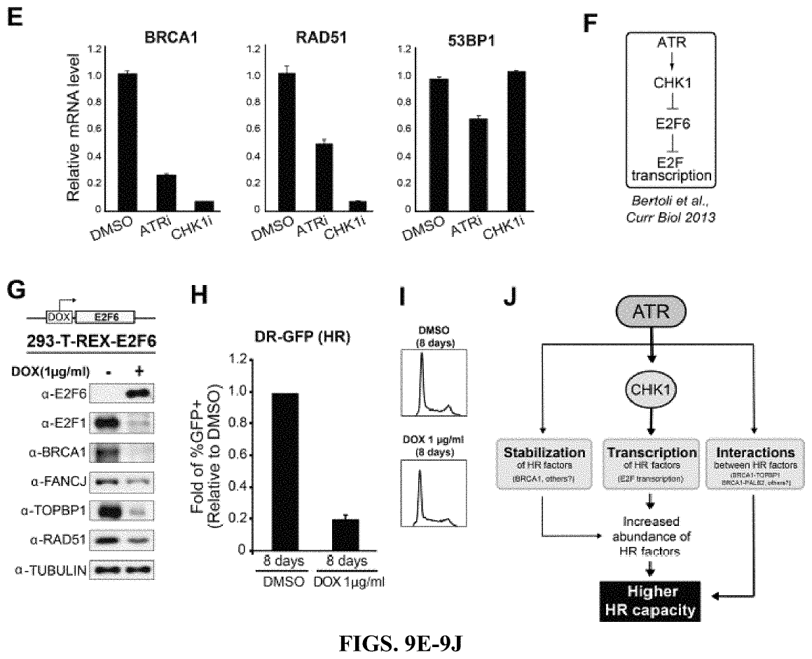

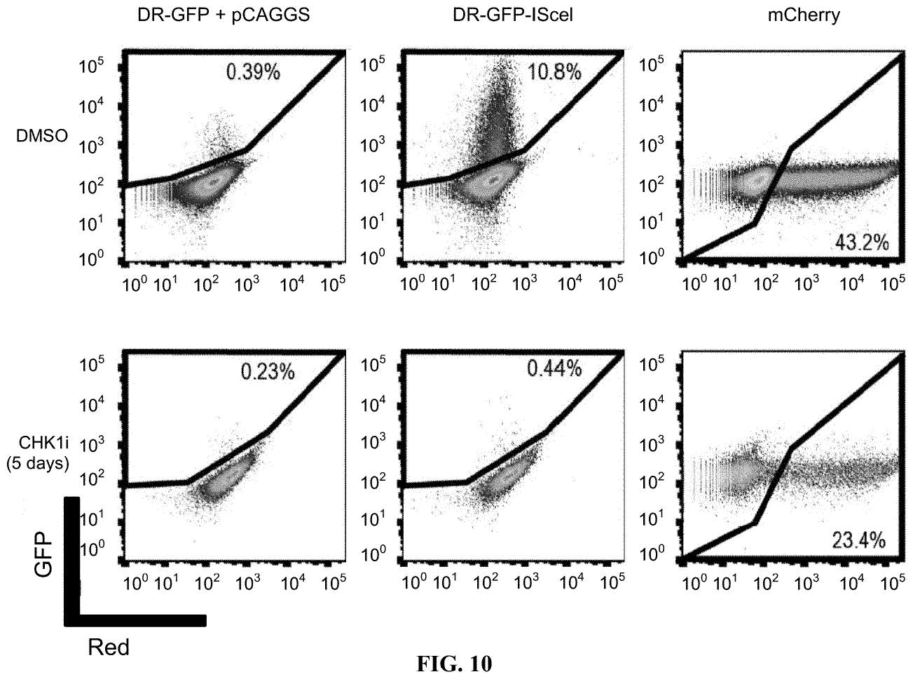

ATR has been shown to control the transcription of E2F targets via activation of the downstream checkpoint kinase CHK1 (Bertoli et al., “Chk1 Inhibits E2F6 Repressor Function in Response to Replication Stress to Maintain Cell-Cycle Transcription,” Curr. Biol. 23:1629-1637 (2013); Buisson et al., “Coupling of Homologous Recombination and the Checkpoint by ATR,” Mol. Cell 65:336-346 (2017); and D'Angiolella et al., “Cyclin F-Mediated Degradation of Ribonucleotide Reductase M2 Controls Genome Integrity and DNA Repair,” Cell 149:1023-1034 (2012), which are hereby incorporated by reference in their entirety). Whether chronic CHK1 inhibition results in a similar depletion of HR factors was observed after chronic ATR inhibition was investigated. As shown in FIG. 9A, chronic CHK1 inhibition severely reduced the abundance of the HR factors BRCA1 and RAD51, as well as the abundance of the canonical E2F target RRM2, and E2F1 itself. The depletion of HR factors after CHK1 inhibition was correlated with a sharp decrease in HR efficiency (FIGS. 9B and 10), and both effects were not a consequence of a reduction in the number of S phase cells since CHK1 inhibition actually results in an increase in the relative number of S phase cells (FIGS. 9C and 9D). Of importance, CHK1 as well as ATR inhibition results in a reduction in the mRNA levels of BRCA1 and RAD51 (FIG. 9E). Taken together, these results are consistent with the model that ATR controls the transcription of key HR factors through the ATR-CHK1 pathway. Notably, major changes in the mRNA levels of 53BP1 were not observed (FIG. 9E), suggesting that ATR inhibition preferentially depletes factors involved in HR, while not changing the protein machinery required for NHEJ, and therefore not impairing NHEJ-mediated repair (FIG. 1B).

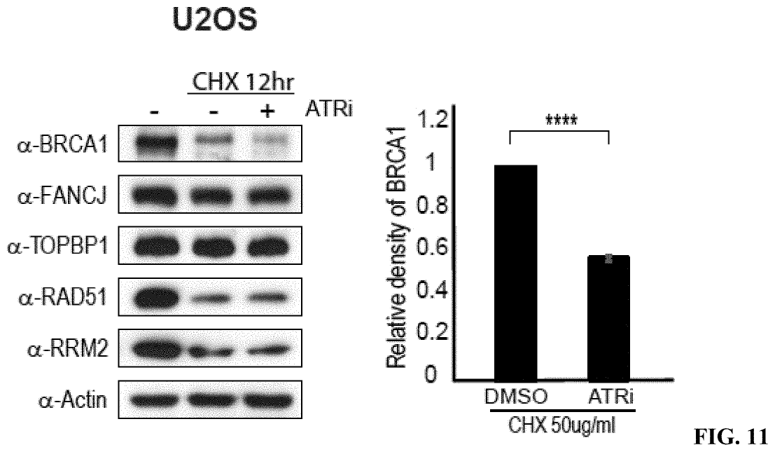

Mechanistically, ATR-CHK1 signaling has been shown to control E2F transcription by inhibiting the E2F6 repressor (FIG. 9F) (Bertoli et al., “Chk1 Inhibits E2F6 Repressor Function in Response to Replication Stress to Maintain Cell-Cycle Transcription,” Curr. Biol. 23:1629-1637 (2013), which is hereby incorporated by reference in its entirety). Therefore, whether chronic overexpression of E2F6 leads to the same effect as ATR inhibition on the HR machinery was investigated. As shown in FIGS. 9G and 9H, a 293-T-REX-E2F6 system was used for doxycycline-induced expression of E2F6 and overexpression of E2F6 was found to result in severe reduction in HR factor abundance and HR capacity. Congruent with these effects not being consequences of differences in cell cycle distribution, no substantial change in the cell cycle upon overexpression of E2F6 was observed (FIG. 9I). Of note, whether the stability of HR proteins can be affected independently of translational control was also examined. U2OS cells were treated with the translation inhibitor cycloheximide (CHX) and whether ATR inhibition results in changes in the abundance of some HR factors was investigated. As shown in FIG. 11, protein levels of BRCA1 are decreased upon CHX treatment and ATR inhibition compared to CHX treatment alone, suggesting that ATR signaling promotes BRCA1 protein stabilization. In U2OS cells, such transcription-independent mode of protein stabilization was only observed for BRCA1 as the abundance of the other HR proteins monitored did not change upon ATR inhibition in CHX-treated cells.

Overall, these findings indicate that ATR controls the abundance of HR proteins through the control of transcription and, in the case of BRCA1, also through the control of protein stability (FIG. 9J). Since BRCA1 has been proposed to be a target of ATR (Liu et al., “TOPBP1(Dpb11) Plays a Conserved Role in Homologous Recombination DNA Repair Through the Coordinated Recruitment of 53BP1(Rad9),” J. Cell Biol. 216: 623-639 (2017); Buisson et al., “Coupling of Homologous Recombination and the Checkpoint by ATR,” Mol. Cell 65:336-346 (2017); and Tibbetts et al., “Functional Interactions Between BRCA1 and the Checkpoint Kinase ATR During Genotoxic Stress,” Genes Dev. 14:2989-3002 (2000), which are hereby incorporated by reference in their entirety), it was hypothesized that ATR-mediated phosphorylation sites in BRCA1 promote BRCA1 stabilization. Overall, the finding indicate that ATR plays multiple pro-HR roles by controlling the abundance of a range of HR proteins and interactions between them (FIG. 9J).

### Example 4—Correlation of HR Factor Abundance and HR Capacity

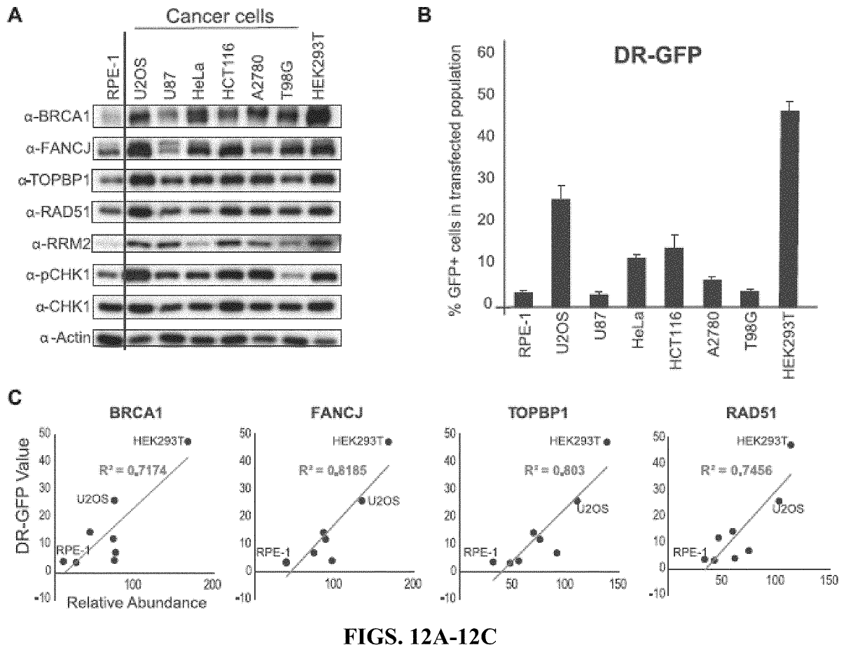

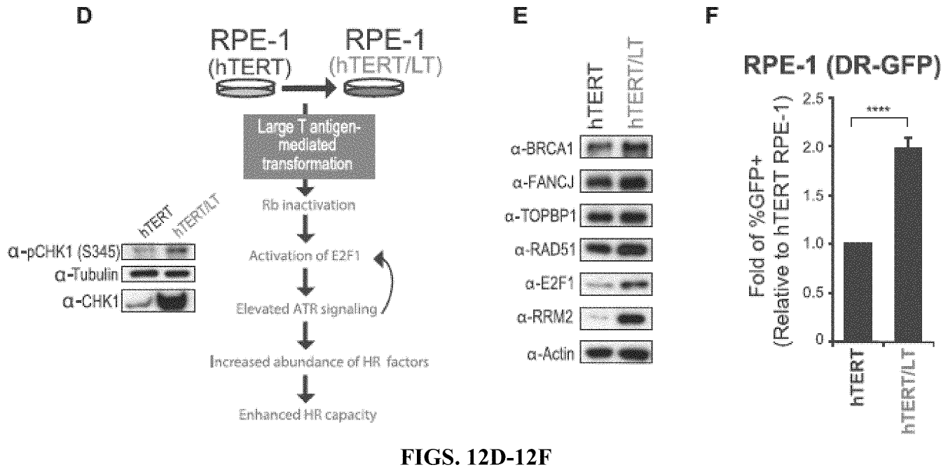

To explore the hypothesis that HR factor abundance is a major determinant of HR capacity, the abundance of selected HR factors (BRCA1, FANCJ, RAD51 and TOPBP1) and HR capacity was monitored in a panel of cell lines. As shown in FIG. 12A, most cancer cells tested had higher levels of the HR factors compared to untransformed hTERT RPE-1 cells, consistent with the fact that increased CHK1 signaling, RB mutation or E2F1 overexpression are often correlated with cancer (Chen et al., “Emerging Roles of E2Fs in Cancer: An Exit from Cell Cycle Control,” Nat. Rev. Cancer 9:785-797 (2009); Sherr et al., “The RB and p53 Pathways in Cancer,” Cancer Cell 2:103-112 (2002); Ma et al., “Overexpression of E2F1 Promotes Tumor Malignancy and Correlates with TNM Stages in Clear Cell Renal Cell Carcinoma,” PLoS One 8:e73436 (2013); Krajewska et al., “ATR Inhibition Preferentially Targets Homologous Recombination-Deficient Tumor Cells,” Oncogene 34:3474-3481 (2015), which are hereby incorporated by reference in their entirety). Analysis of HR using DR-GFP assay revealed major differences in HR capacity in the cells monitored (FIG. 12B). While hTERT RPE-1 cells had the lowest level of HR factors and displayed the lowest HR capacity, HEK293T cells had the highest level of HR factors and displayed the highest HR capacity (FIGS. 12A and 12B). Overall, there was a reasonable correlation between HR capacity and the abundance of HR factors (FIG. 12C). These findings are consistent with a previous report showing that untransformed hESC have lower HR efficiency than cancer cells (Fung et al., “Repair at Single Targeted DNA Double-Strand Breaks in Pluripotent and Differentiated Human Cells,” PLoS One 6:e20514 (2011), which is hereby incorporated by reference in its entirety).

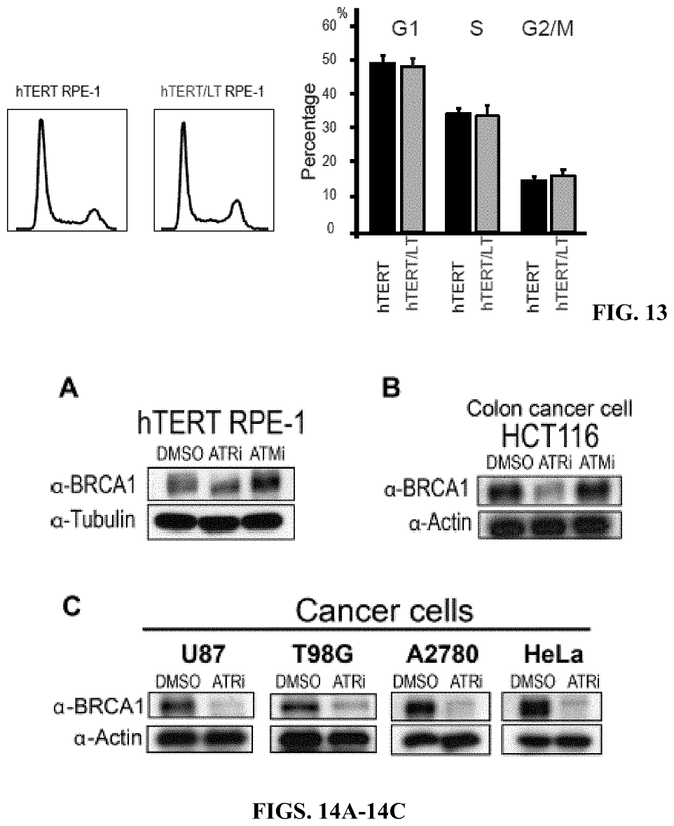

Consistent with the hypothesis that E2F transcription impacts the level of HR-mediated repair, hTERT RPE-1 cells transformed with Large T (LT) antigen (FIG. 12D), which was previously shown to result in high expression of E2F targets via impairment of RB function (Ahuja et al., “SV40 Large T Antigen Targets Multiple Cellular Pathways to Elicit Cellular Transformation,” Oncogene 24:7729-7745 (2005) and Cantalupo et al., “Cell-Type Specific Regulation of Gene Expression by Simian Virus 40 T Antigens,” Virology 386:183-191 (2009), which are hereby incorporated by reference in their entirety), resulted in increased HR factor abundance and HR capacity (FIGS. 12E and 12F). It has also been reported that LT antigen transformation and E2F1 overexpression can activate the ATR/ATM pathway (Rohaly et al., “Simian Virus 40 Activates ATR-Delta p53 Signaling to Override Cell Cycle and DNA Replication Control,” J. Virol. 84:10727-10747 (2010); Forero et al., “Simian Virus 40 Large T Antigen Induces IFN-Stimulated Genes Through ATR Kinase,” J. Immunol. 192:5933-5942 (2014); and Powers et al., “E2F1 uses the ATM Signaling Pathway to Induce p53 and Chk2 Phosphorylation and Apoptosis,” Mol. Cancer Res. 2:203-214 (2004), which are hereby incorporated by reference in their entirety), and as expected, phospho-CHK1 levels increased upon LT transformation, suggesting that ATR is more active in LT transformed cells (FIG. 12D). LT transformation did not result in major cell cycle changes, confirming that the protein abundance changes are not a cell cycle effect (FIG. 13). Taken together, these findings are consistent with the model that HR factor abundance is a key determinant of HR capacity and further supports the importance of the role of ATR signaling in controlling HR capacity via modulation of HR factor abundance.

### Example 5—Cancer Cell Lines Heavily Rely on ATR to Sustain the Abundance of HR Factors

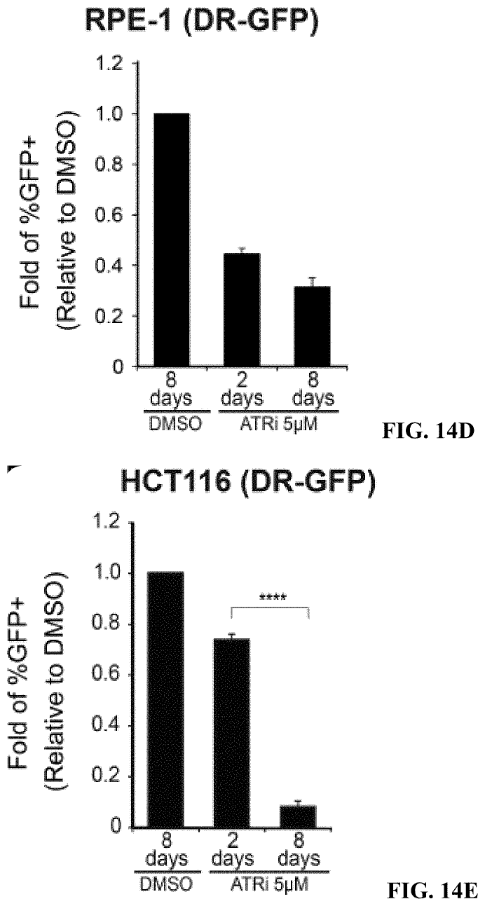

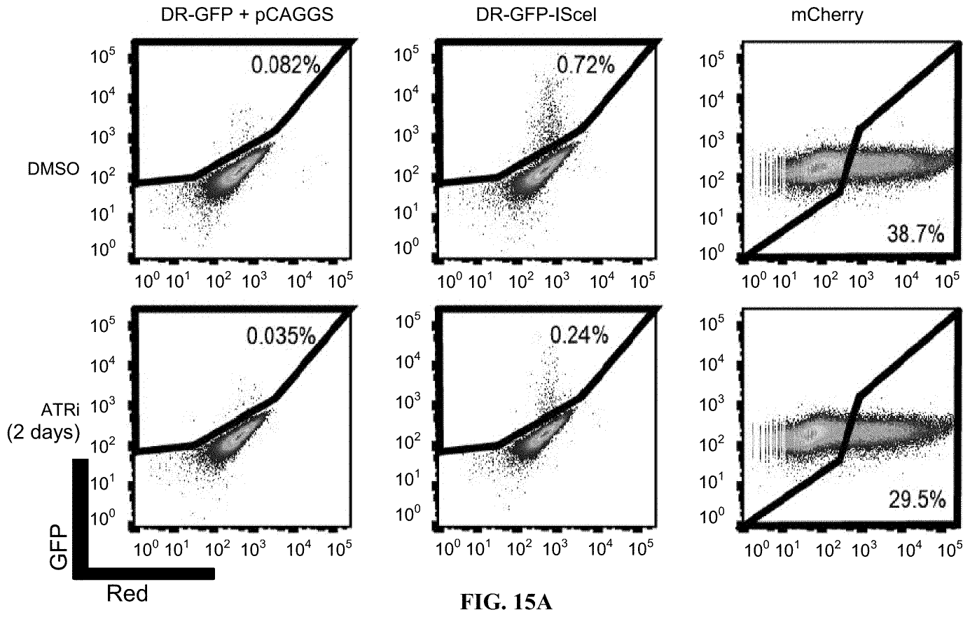

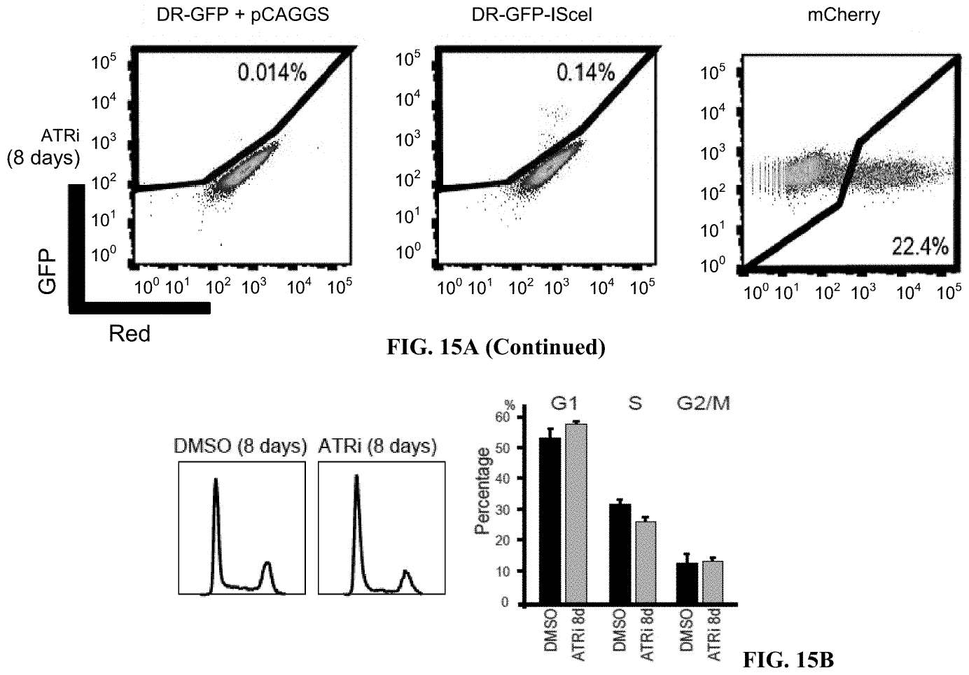

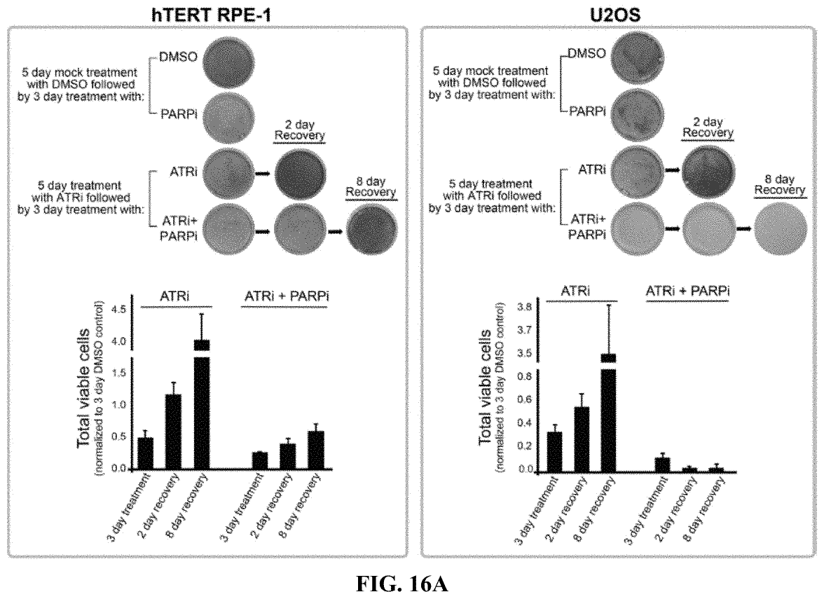

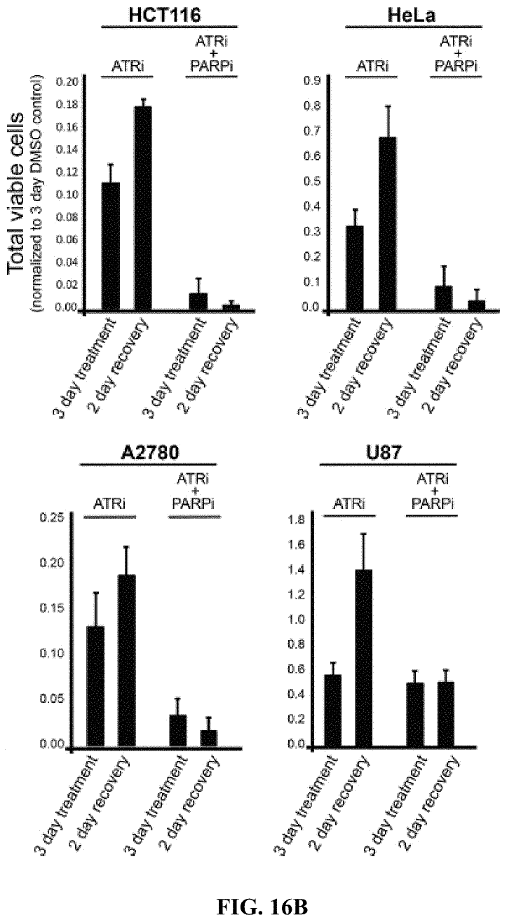

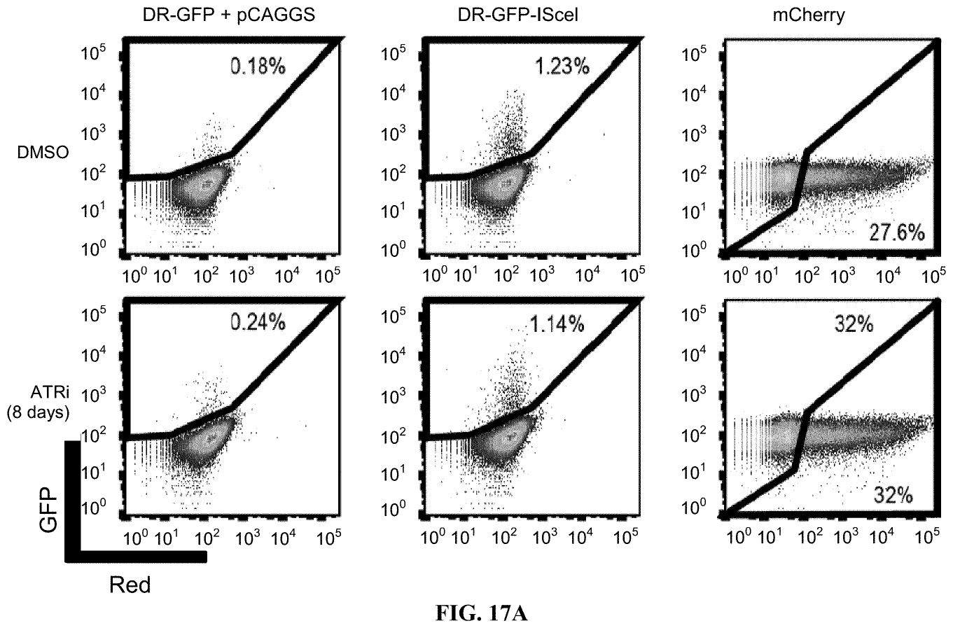

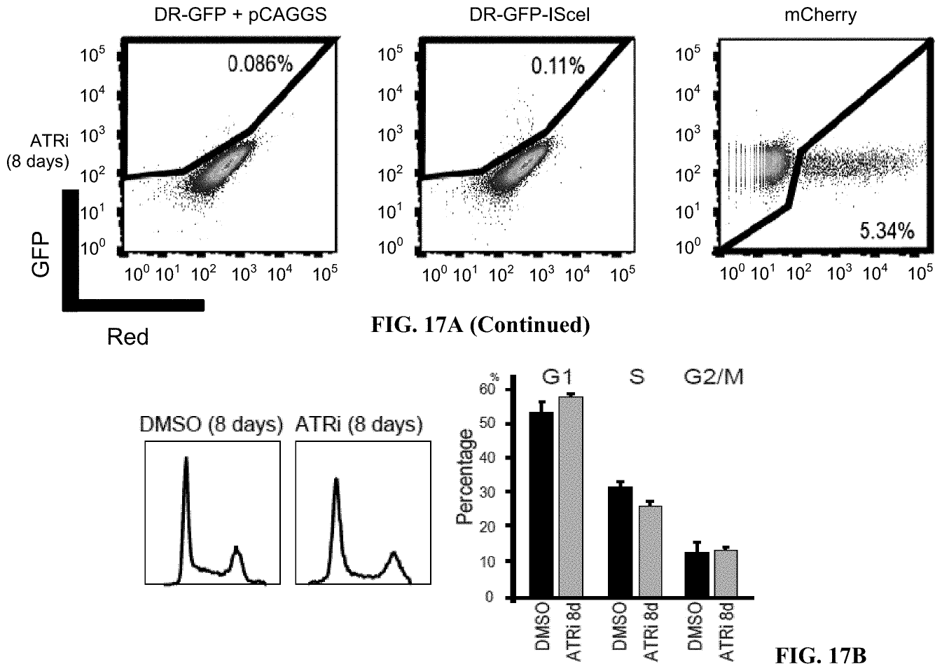

Many cancer cells are known to have de-regulated DNA replication and, as a consequence, higher levels of replication stress Hook et al., “Mechanisms to Control Rereplication and Implications for Cancer,” Curr. Opin. Cell Biol. 19:663-671 (2007); Blow et al., “Replication Licensing and Cancer—A Fatal Entanglement?,” Nat. Rev. Cancer 8:799-806 (2008); and Halazonetis et al., “An Oncogene-Induced DNA Damage Model for Cancer Development,” Science 319:1352-1355 (2008), which are hereby incorporated by reference in its entirety). Since ATR signaling is responsive to intrinsic levels of replication stress, it was reasoned that higher levels of basal ATR signaling in cancer cells result in increased abundance of HR factors and higher HR capacity. Consistent with this model, it was proposed that cells become addicted to E2F transcription to cope with high levels of replication stress (Bertoli et al., “Sustained E2F-Dependent Transcription Is a Key Mechanism to Prevent Replication-Stress-Induced DNA Damage,” Cell Rep. 15:1412-1422 (2016), which is hereby incorporated by reference in its entirety). As shown in FIG. 14A, phospho-CHK1 and the abundance of key HR proteins were indeed higher in a panel of cancer cell lines compared to an hTERT RPE-1 cell typically used as a non-cancer cell line reference. It was predicted that these cancer cell lines would be highly dependent on ATR signaling to maintain the abundance of HR proteins. Indeed, while chronic ATRi treatment did not result in major changes in the abundance of BRCA1 in hTERT RPE-1 cells (FIG. 13A), severe reduction in the abundance of BRCA1 was found in cancer cells (FIGS. 14B and 14C). Consistent with this finding, chronic ATR inhibition in hTERT RPE-1 cells did not result in a striking drop in HR-mediated repair efficiency compared to short-term (2 day) treatment (FIGS. 14D and 15), whereas in the HCT116 colon cancer cell line, the effect of chronic ATR inhibition was significantly more severe than that of short-term treatment (FIGS. 16E and 17). Taken together, the results reveal cell type-specific responses to long-term ATR inhibition, with cancer cells often displaying stronger dependency on ATR signaling for sustaining the abundance of HR factors. The sharp correlation between observed changes in HR protein abundance and measured changes in HR-mediated repair efficiency strongly suggests that reduction in HR factor abundance is the major cause of the reduction in HR-mediated repair efficiency.

### Example 6—Rationale for Synergistic Sensitivity of Cancer Cells to Combined ATR and PARP Inhibition

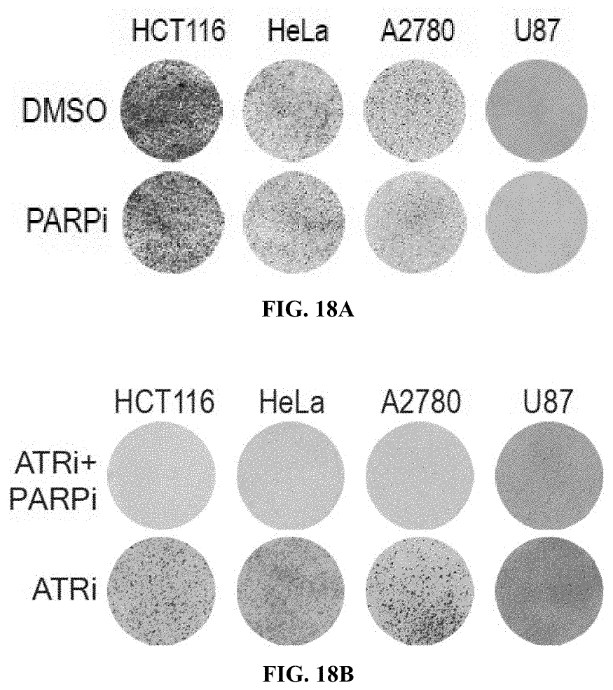

The results here reveal that chronic ATR inhibition mimics “BRCAness”, a term used to define a state of cancer cell lines with dysfunctional BRCA1 or BRCA2, and therefore, dysfunctional HR-mediated repair (Turner et al., “Hallmarks of ‘BRCAness’ in Sporadic Cancers,” Nat. Rev. Cancer 4:814-819 (2004), which is hereby incorporated by reference in its entirety). Of note, BRCAness cells with BRCA1 or BRCA2 mutations are highly sensitive to PARP inhibitors (Bryant et al., “Specific Killing of BRCA2-Deficient Tumours with Inhibitors of Poly(ADP-ribose) polymerase,” Nature 434:913-917 (2005); Farmer et al., “Targeting the DNA Repair Defect in BRCA Mutant Cells as a Therapeutic Strategy,” Nature 434:917-921 (2005); Rottenberg et al., “High Sensitivity of BRCA1-Deficient Mammary Tumors to the PARP Inhibitor AZD2281 Alone and in Combination with Platinum Drugs,” Proc. Natl. Acad. Sci. USA 105:17079-17084 (2008); and Fong et al., “Inhibition of Poly(ADP-Ribose) Polymerase in Tumors from BRCA Mutation Carriers,” N. Engl. J. Med. 361:123-134 (2009), which are hereby incorporated by reference in its entirety), which are clinically effective drugs under FDA approval for ovarian and breast cancer treatment. It was therefore reasoned that long-term treatment with ATRi could be used to hypersensitize HR-proficient cancer cells to PARP inhibitors. Based on this rationale, cells should first undergo a long-term treatment with a low dose of ATRi to deplete key HR components (such low dose would have a minor effect on non-cancer cells), and PARP inhibitor should only be added after this first treatment phase. In this manner, depletion of the HR machinery in HR-proficient cancer cells will make them particularly sensitive to a second treatment phase in which PARP inhibitors are also added. To test the efficacy of this strategy, hTERT RPE-1 and HR-proficient U2OS cancer cells were subjected to 5 days of chronic ATRi treatment. These cells were then with ATR and/or PARP inhibitors for an additional 3 days (FIG. 16A). Crystal violet cell staining assay showed that hTERT RPE-1 cells did not show major changes after inhibitor treatment, whereas U2OS cancer cells showed dramatic loss of viability upon combined treatment with ATR and PARP inhibitors. Strikingly, U2OS cells treated only with ATRi for 8 days recovered after switching to drug-free media, whereas U2OS cells undergoing the full treatment regime including the combination of ATRi and PARPi, could not recover. Interestingly, other cancer cells that have relatively higher HR capacity compared to hTERT RPE-1 cells (FIG. 12B), including HeLa, HCT116 and A2780, also did not recover well after the full ATRi/PARPi combination treatment (FIGS. 16B, 18). Of note, U87 cancer cells did not recover as well as hTERT-RPE-1 cells, but for reasons that remain unknown, seem not as impacted as the other cancer cells by the combined ATRi/PARPi treatment. Overall, the described findings are consistent with previous reports that showed synthetic lethality of ATR and PARP inhibition in cancer cells and tumors (Mohni et al., “A Synthetic Lethal Screen Identifies DNA Repair Pathways that Sensitize Cancer Cells to Combined ATR Inhibition and Cisplatin Treatments,” PLoS One 10:e0125482 (2015) and Kim et al., “Targeting the ATR/CHK1 Axis with PARP Inhibition Results in Tumor Regression in BRCA-Mutant Ovarian Cancer Models,” Clinical Cancer Research 23:3097-3108 (2017), which are hereby incorporated by reference in its entirety). Collectively, the results presented herein provide the mechanistic explanation for why the ATRi/PARPi combination can be so effective at sensitizing HR-proficient cancer cells.

### Discussion of Examples 1-6

The work presented herein reveals a major role for ATR signaling in modulating HR capacity by controlling of the abundance of the recombination machinery. These results have important implications to understand how cancer cells acquire enhanced HR capacity. Furthermore, the reported findings provide rationale for the therapeutic use of ATR inhibitors in sensitizing HR-proficient cancer cells to drugs known to target HR-deficient cancer cells.

### A Model for the Control of HR Capacity Via ATR Signaling

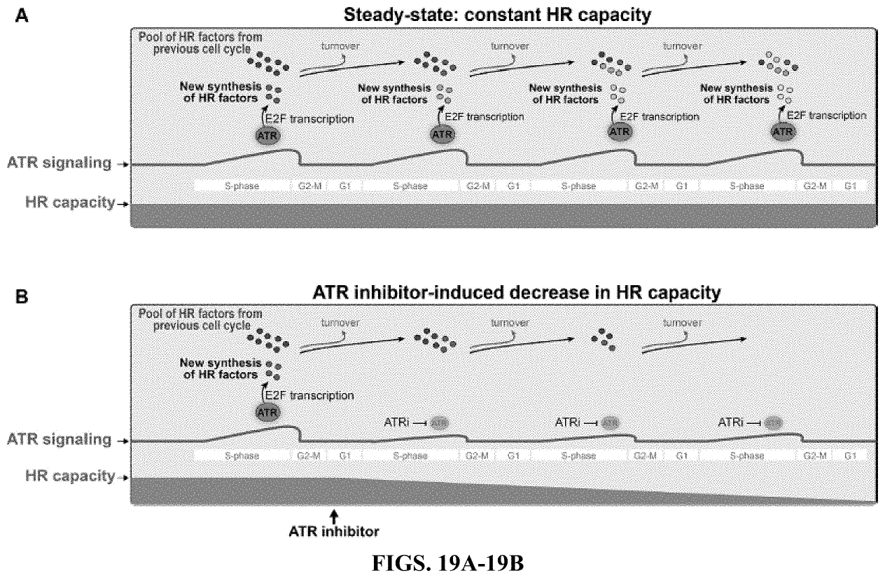

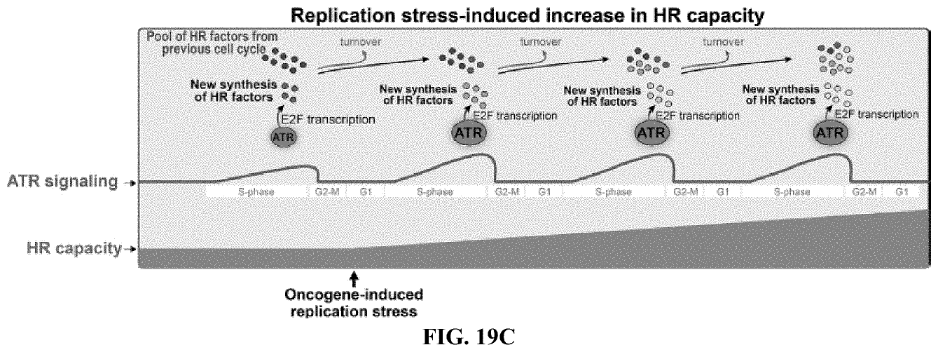

ATR signaling mediates key interactions between HR proteins that are important for HR-mediated repair (Liu et al., “TOPBP1(Dpb11) Plays a Conserved Role in Homologous Recombination DNA Repair Through the Coordinated Recruitment of 53BP1(Rad9),” J. Cell Biol. 216:623-639 (2017); Buisson et al., “Coupling of Homologous Recombination and the Checkpoint by ATR,” Mol. Cell 65:336-346 (2017); and Dubois et al., “A Phosphorylation-and-Ubiquitylation Circuitry Driving ATR Activation and Homologous Recombination,” Nucleic Acids Res. 45:8859-8872 (2017), which are hereby incorporated by reference in their entirety). In this context, short-term inhibition of ATR is expected to impair such pro-HR function of ATR. Indeed, the modest reduction in HR observed upon short-term ATR inhibition may be attributed to loss of specific key pro-HR interactions (for example: BRCA1-TOPBP1 and PALB2-BRCA1), although it is possible that ATM may partially compensate for the loss of ATR signaling in mediating these interactions. While it is possible that the impairment of protein interactions contributes partially to the severe inhibition of HR observed upon long-term (over 4 days) ATR inhibition, the model that the severe reduction in HR capacity is mostly caused by a pre-conditioned state of HR factor depletion is favored. In this model, the role of ATR in controlling transcription of E2F targets during every S-phase is crucial to maintain the proper abundance of HR proteins and, therefore, sustain the capacity of cells to utilize HR (FIGS. 19A-19C). Consistent with this model, E2F targets, which include many HR factors, are induced every S phase as part of a G1/S wave of cell cycle transcription (Bertoli et al., “Control of Cell Cycle Transcription During G1 and S Phases,” Nat. Rev. Mol. Cell Biol. 14:518-528 (2013) and Bracken et al., “E2F target genes: unraveling the biology,” Trends Biochem. Sci. 29:409-417 (2004), which are hereby incorporated by reference in their entirety). Furthermore, the ATR-CHK1 pathway is supposedly active every S-phase (Bastos de Oliveira et. al., “Phosphoproteomics Reveals Distinct Modes of Mec1/ATR Signaling During DNA Replication,” Mol. Cell 57:1124-1132 (2015); Sorensen et al., “Safeguarding Genome Integrity: The Checkpoint Kinases ATR, CHK1 and WEE1 Restrain CDK Activity During Normal DNA Replication,” Nucleic Acids Res. 40:477-486 (2012); and Enders et al., “Expanded Roles for Chk1 in Genome Maintenance,” J. Biol. Chem. 283:17749-17752 (2008), which are hereby incorporated by reference in their entirety) and is known to prolong E2F transcription (Bertoli et al., “Control of Cell Cycle Transcription During G1 and S Phases,” Nat. Rev. Mol. Cell Biol. 14:518-528 (2013); Bertoli et al., “Sustained E2F-Dependent Transcription Is a Key Mechanism to Prevent Replication-Stress-Induced DNA Damage,” Cell Rep. 15:1412-1422 (2016); Bertoli et al., “Chk1 Inhibits E2F6 Repressor Function in Response to Replication Stress to Maintain Cell-Cycle Transcription,” Curr. Biol. 23:1629-1637 (2013); Herlihy et al., “The Role of the Transcriptional Response to DNA Replication Stress,” Genes (Basel) 8 (2017); Bastos de Oliveira et al., “Linking DNA Replication Checkpoint to MBF Cell-Cycle Transcription Reveals a Distinct Class of G1/S Genes,” EMBO J. 31:1798-1810 (2012); and Travesa et al., “DNA Replication Stress Differentially Regulates G1/S Genes via Rad53-Dependent Inactivation of Nrm1,” EMBO J. 31:1811-1822 (2012), which are hereby incorporated by reference in their entirety). Therefore, the work presented herein suggests that chronic ATR inhibition during multiple cell cycles gradually decreases the abundance of HR factors (FIG. 19B). A logical explanation is that under chronic ATR inhibition the amount of protein loss due to constitutive protein turnover surpasses the amount of HR factors produced by de novo protein synthesis, generating a deficit in HR factor abundance that is exacerbated after multiple cell cycles. As a consequence, depletion of the HR machinery severely impairs HR capacity. While it is currently unclear whether the level of a specific HR factor or global levels that are behind the synergistic sensitivity to ATRi/PARPi, the model that the strong reduction in HR-mediated repair is due to the combined reduction in the abundance of several repair factors is favored.

### Increased ATR Signaling Enhances the HR Capacity in Cancer Cells

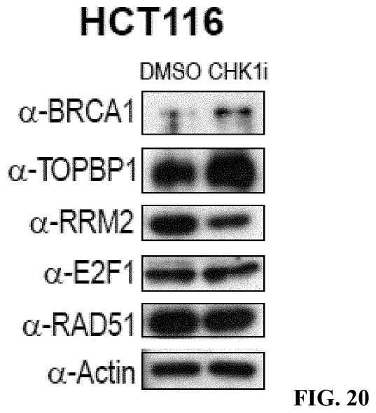

Many cancer cells experience high levels of replication stress (Hills et al., “DNA Replication and Oncogene-Induced Replicative Stress,” Current Biology 24:R435-R444 (2014); Halazonetis et al., “An Oncogene-Induced DNA Damage Model for Cancer Development,” Science 319:1352-1355 (2008); Bartek et al., “Thresholds of Replication Stress Signaling in Cancer Development and Treatment,” Nat. Struct. Mol. Biol. 19:5-7 (2012); Macheret et al., “DNA Replication Stress as a Hallmark of Cancer,” Annu. Rev. Pathol-Mech. 10:425-448 (2015); and Murga et al., “Exploiting Oncogene-Induced Replicative Stress for the Selective Killing of Myc-Driven Tumors,” Nat. Struct. Mol. Biol. 18:1331-1335 (2011), which are hereby incorporated by reference in their entirety), which results in higher levels of spontaneous ATR signaling (Herlihy et al., “The Role of the Transcriptional Response to DNA Replication Stress,” Genes (Basel) 8 (2017); Flynn et al., “ATR: A Master Conductor of Cellular Responses to DNA Replication Stress,” Trends Biochem. Sci. 36:133-140 (2011); and Marechal et al., “DNA Damage Sensing by the ATM and ATR Kinases,” Cold Spring Harb. Perspect. Biol. 5 (2013), which are hereby incorporated by reference in their entirety). Based on the model presented in FIG. 19C, applicant concludes that such constitutively higher ATR signaling in cancer cells during every S-phase will create a surplus in HR factor abundance and lead to higher HR capacity. In fact, many cancers have increased HR capacity (Fung et al., “Repair at Single Targeted DNA Double-Strand Breaks in Pluripotent and Differentiated Human Cells,” PLoS One 6:e20514 (2011), which is hereby incorporated by reference in its entirety). It is worth mentioning that a correlation between ATR signaling and HR capacity could be lost due to mutations or alterations that directly deregulate the RB-E2F pathway. For example, the results presented herein demonstrate that HEK293T cells exhibit one of the highest levels of HR factors and the highest HR capacity of all cells examined, but not the highest levels of CHK1 phosphorylation (FIGS. 12A and 12B). This is consistent with the fact that LT-transformation is known to directly inhibit RB function, and therefore maintain high E2F transcription (Ahuja et al., “SV40 Large T Antigen Targets Multiple Cellular Pathways to Elicit Cellular Transformation,” Oncogene 24:7729-7745 (2005) and Cantalupo et al., “Cell-Type Specific Regulation of Gene Expression by Simian Virus 40 T Antigens,” Virology 386:183-191 (2009), which are hereby incorporated by reference in their entirety), which would bypass the need for CHK1 signaling for enhanced HR factor abundance. Interestingly, the potential for an ATR-dependent and CHK1-independent mechanism for HR factor control in HCT116 cells was also noted. As shown in FIG. 20, differently than ATR inhibition, CHK1 inhibition did not lower the level of E2F1 nor the abundance of other HR proteins in HCT116. This result suggests that there is an alternative pathway, other than CHK1, that regulates E2F transcription in these cells. Finally, that ATR may also control BRCA1 protein stability, revealing transcription-independent mechanisms for ATR-dependent regulation of HR factor abundance is also noted. Overall, despite possibilities for alternative rewiring of the mechanisms for control of E2F transcription, the model for enhanced ATR signaling promoting increased HR factor abundance seems applicable in many cases and provides a mechanistic explanation for the high HR capacity observed in many cancers.

### A Chemo-Therapeutic Strategy to Sensitize HR Proficient Cancer Cells to PARP Inhibitors

The finding that chronic treatment of cancer cells with sub-lethal doses of ATRi leads to severe impairment of HR-mediated repair has important implications for cancer therapy. As described herein above, many cancer cells seem to rely on constitutive ATR hyper-signaling to over-express components of the HR machinery and acquire enhanced HR capacity, which would improve their ability to deal with increased levels of replication stress. In this context, applicant proposes that partial inhibition of ATR over long-term treatment protocol is particularly deleterious for cancer cells, because the compromised HR machinery is expected to be unable to efficiently deal with enhanced replication stress. Of importance, since the treatment uses relatively low doses of ATRi, it is possible that cells such as hTERT RPE-1 (and potentially other non-cancer cells) that do not explore ATR hyper-signaling, are not subjected to the deleterious effects of a high dose of ATR or CHK1 inhibitor, which include the well-established effects on origin firing and fork integrity.

Since chronic ATR inhibition is very effective at depleting the HR machinery and reducing HR capacity, applicant further predicts that this rationale could be explored in combination therapy to sensitize HR-proficient cancer cells to the treatment of PARP inhibitors, which are typically used to treat HR-deficient cancers such as BRCA1 or BRCA2 mutated cancers (Bryant et al., “Specific Killing of BRCA2-Deficient Tumours with Inhibitors of Poly(ADP-ribose) Polymerase,” Nature 434:913-917 (2005); Farmer et al., “Targeting the DNA Repair Defect in BRCA Mutant Cells as a Therapeutic Strategy,” Nature 434:917-921 (2005); Rottenberg et al., “High Sensitivity of BRCA1-Deficient Mammary Tumors to the PARP Inhibitor AZD2281 Alone and in Combination with Platinum Drugs,” Proc. Natl. Acad. Sci. USA 105:17079-17084 (2008); Fong et al., “Inhibition of Poly(ADP-ribose) Polymerase in Tumors from BRCA Mutation Carriers,” N. Engl. J. Med. 361:123-134 (2009), which are hereby incorporated by reference in their entirety). Indeed, the data on a panel of cancer lines presented herein revealed that the prediction is correct for most of the cancer cell lines tested. Further analyses involving a larger panel of cancer cell lines and in organismal contexts will be required to further evaluate the effectiveness of this approach. A recent study showed that ATR inhibition and knockdown of HR factors, such as RAD51, leads to synergistic lethality in cancer cells (Krajewska et al., “ATR Inhibition Preferentially Targets Homologous Recombination-Deficient Tumor Cells,” Oncogene 34:3474-3481 (2015), which is hereby incorporated by reference in its entirety). While this is an interesting and potentially effective strategy, it is likely that this synergism arises from the short-term effects of ATR inhibitors in causing fork collapse, which would then require the HR machinery for fork restart. In this context, ATR inhibitors are being used as a DNA damage-generating drug. Such a rationale is fundamentally distinct from the rationale presented herein, in which treatment with sub-lethal doses of ATR inhibitors is used to reduce HR capacity and compromise the ability of cancer cells to respond to DNA damage.

Overall, the findings reported herein provide a novel strategy for how the HR capacity can be modulated in cancer cells via controlled ATR inhibition. In addition to helping improve therapy, it is expected that the generated knowledge could have far reaching implications to better understand how cancer cells develop an optimized machinery for robust genome replication and maintenance.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of sensitizing homologous recombination (HR)-proficient cancer cells to treatment with poly ADP ribose polymerase (PARP) inhibitors, said method comprising:
providing HR-proficient cancer cells and
treating the HR-proficient cancer cells with an Ataxia telangiectasia and Rad3-related (ATR) inhibitor under conditions effective to sensitize the HR-proficient cancer cells to treatment with PARP inhibitors, wherein said treating is carried out without administering PARP inhibitors.

2. The method of claim 1, wherein said treating is carried out daily for at least 5-7 days.

3. The method of claim 1, wherein said ATR inhibitor is VE-822 or AZD6738.

4. The method of claim 3, wherein said treating is carried out daily with a concentration of ATR inhibitor of 0.125 to 0.25 μM.

5. The method of claim 1, wherein said ATR inhibitor is VE-821.

6. The method of claim 5, wherein said treating is carried out daily with a concentration of ATR inhibitor of 2.5 to 5.0 μM.

7. The method of claim 1, wherein the cancer cells are selected from the group consisting of osteosarcoma cells, colon cancer cells, ovarian cancer cells, cervical cancer cells, glioblastoma cells, neuroblastoma cells, lung cancer cells, and pancreatic cancer cells.

8. The method of claim 1, wherein said sensitizing converts HR-proficient cancer cells to HR-deficient cancer cells.

9. A method of treating cancer in a subject, said method comprising:
selecting a subject with a cancer mediated by homologous recombinant (HR)-proficient cells and
treating the selected subject with an Ataxia telangiectasia and Rad3-related (ATR) inhibitor under conditions effective to sensitize the HR-proficient cancer cells to treatment with PARP inhibitors, wherein said treating is carried out without administering PARP inhibitors.

10. The method of claim 9, where said administering is carried out daily for at least 5-7 days.

11. The method of claim 9, wherein said ATR inhibitor is VE-822 or AZD6738.

12. The method of claim 11, wherein said treating is carried out daily at a dose of ATR inhibitor of 0.5 to 4 milligrams per the subject's mass in kilograms.

13. The method of claim 9, wherein said ATR inhibitor is VE-821.

14. The method of claim 13, wherein said treating is carried out daily at a dose of 5 to 80 milligrams per the subject's mass in kilograms.

15. The method of claim 9, wherein the cancer is selected from the group consisting of osteosarcoma, colon cancer, ovarian cancer, cervical cancer, glioblastoma, neuroblastoma, lung cancer, and pancreatic cancer.

16. The method of claim 9, wherein said sensitizing converts HR-proficient cancer cells to HR-deficient cancer cells.

17. A method of treating HR-proficient cancer cells, said method comprising:
providing HR-proficient cancer cells;
treating the HR-proficient cancer cells with an ATR inhibitor, wherein said treating is carried out without administering PARP inhibitors; and
administering to the treated HR-proficient cancer cells an ATR inhibitor and a PARP inhibitor.

18. The method of claim 17, wherein said treating is carried out under conditions insufficient to kill cells and wherein said administering is carried out under conditions to kill cancer cells but insufficient to kill non-cancer cells.

19. The method of claim 17, wherein said treating is carried out daily for at least 5-7 days and said administering is carried out daily for at least 2-3 days.

20. The method of claim 17, wherein said ATR inhibitor is VE-822 or AZD6738.

21. The method of claim 20, wherein said treating is carried out daily with a concentration of ATR inhibitor of 0.125 to 0.25 μM.

22. The method of claim 17, wherein said ATR inhibitor is VE-821.

23. The method of claim 22, wherein said treating is carried out daily with a concentration of ATR inhibitor of 2.5 to 5.0 μM.

24. The method of claim 17, wherein said administering is carried out with a PARP inhibitor selected from the group consisting of olaparib (AZD 2281), rucaparib (AG 014699), niraparib (MK 4827), talozaparib (BMN 673), AZD 2461, and veliparib (ABT-888).

25. The method of claim 24, wherein the PARP inhibitor is administered daily at a concentration of 2.5 to 10 μM.

26. The method of claim 17, wherein the cancer cells are selected from the group consisting of osteosarcoma cells, colon cancer cells, ovarian cancer cells, cervical cancer cells, glioblastoma cells, neuroblastoma cells, lung cancer cells, and pancreatic cancer cells.

27. A method of treating a cancer patient, said method comprising:
selecting a subject with a cancer mediated by HR-proficient cancer cells;
treating the selected subject with an ATR inhibitor, wherein said treating is carried out without administering PARP inhibitors; and
administering to the selected subject an ATR inhibitor and a PARP inhibitor.

28. The method of claim 27, wherein said treating is carried out under conditions insufficient to kill cells and wherein said administering is carried out under conditions to kill cancer cells but insufficient to kill non-cancer cells.

29. The method of claim 27, wherein said treating is carried out daily for at least 5-7 days and said administering is carried out daily for at least 2-3 days.

30. The method of claim 27, wherein said ATR inhibitor is VE-822 or AZD6738.

31. The method of claim 30, wherein said treating is carried out daily at a dose of ATR inhibitor of 0.5 to 4 milligrams per the subject's mass in kilograms.

32. The method of claim 27, wherein said ATR inhibitor is VE-821.

33. The method of claim 32, wherein said treating is carried out daily at a dose of ATR inhibitor of 5 to 80 milligrams per the subject's mass in kilograms.

34. The method of claim 27, wherein the cancer is selected from the group consisting of osteosarcoma, colon cancer, ovarian cancer, cervical cancer, glioblastoma, neuroblastoma, lung cancer, and pancreatic cancer.

35. The method of claim 27, wherein said administering is carried out with a PARP inhibitor selected from the group consisting of olaparib (AZD 2281), rucaparib (AG 014699), niraparib (MK 4827), talozaparib (BMN 673), AZD 2461, and veliparib (ABT-888).

36. The method of claim 27, wherein the PARP inhibitor is administered daily at a concentration of 2.5 to 10 μM grams per the subject's mass in kilograms.


SEQUENCE LISTING (8 sequences)

SEQ ID NO: 1 - DNA, 22 bp, Artificial Sequence
  BRCA1-F primer
accttggaactgtgagaactct

SEQ ID NO: 2 - DNA, 23 bp, Artificial Sequence
  BRCA1-R primer
tcttgatctcccacactgcaata

SEQ ID NO: 3 - DNA, 22 bp, Artificial Sequence
  RAD51-F primer
caacccatttcacggttagagc

SEQ ID NO: 4 - DNA, 21 bp, Artificial Sequence
  RAD51-R primer
ttctttggcgcataggcaaca

SEQ ID NO: 5 - DNA, 21 bp, Artificial Sequence
  53BP1-F primer
atggaccctactggaagtcag

SEQ ID NO: 6 - DNA, 22 bp, Artificial Sequence
  53BP1-R primer
tttctttgtgcgtctggagatt

SEQ ID NO: 7 - DNA, 24 bp, Artificial Sequence
  GAPDH-F primer
tcattgacctcaactacatggttt

SEQ ID NO: 8 - DNA, 21 bp, Artificial Sequence
  GAPDH-R primer
ggaagatggtgatgggatttc